(19) United States
(12) Patent Application Publication
(10) Pub. No.: US 2024/0216542 A1
(43) Pub. Date: Jul. 4, 2024

(54) GM3 SYNTHASE VECTORS AND USES THEREOF

(71) Applicants: University of Massachusetts, Boston, MA (US); The Clinic for Special Children, Inc., Strasburg, PA (US)

(72) Inventors: Huiya Yang, Worcester, MA (US); Guangping Gao, Worcester, MA (US); Kevin A. Strauss, Strasburg, PA (US); Dan Wang, Worcester, MA (US)

(73) Assignees: University of Massachusetts, Boston, MA (US); The Clinic for Special Children, Inc., Strasburg, PA (US)

(21) Appl. No.: 18/557,102

(22) PCT Filed: Apr. 18, 2022
(86) PCT No.: PCT/US2022/025208

§ 371 Date: Oct. 25, 2023

Related U.S. Application Data
(60) Provisional application No. 63/179,684, filed on Apr. 26, 2021.

(51) Int. Cl. A61K 48/00; A61P 3/00; A61P 25/00; C12N 9/10; C12N 15/86

(52) U.S. Cl. CPC A61K 48/0058; A61K 48/0075; A61P 3/00; A61P 25/00; C12N 9/1081; C12N 15/86; C12Y 204/99009; C12N 2750/14143; C12N 2830/008

(57) ABSTRACT

Aspects of the disclosure relate to methods for expressing one or more Ganglioside GM3 synthase (GM3S) isoforms in a cell or subject. In some aspects, the disclosure relates to methods for treating GM3 synthase deficiency in a subject in need thereof by administering an rAAV expressing one or more Ganglioside GM3 synthase (GM3S) isoforms via intracerebroventricular (ICV) administration.

### RELATED APPLICATIONS

This application is a national stage filing under 35 U.S.C. § 371 of international PCT application PCT/US2022/025208, filed Apr. 18, 2022, which claims priority under 35 U.S.C. § 119(e) to U.S. provisional patent application, U.S. Ser. No. 63/179,684, filed Apr. 26, 2021, the entire contents of each of which are incorporated by reference herein.

### REFERENCE TO AN ELECTRONIC SEQUENCE LISTING

The contents of the electronic sequence listing (U012070158US01-SEQ-KZM.txt; Size: 255,512 bytes; and Date of Creation: Oct. 2, 2023) are herein incorporated by reference in their entirety.

### BACKGROUND

GM3 synthase (GM3S) deficiency is a rare monogenic neurological disorder common within Old Order Amish communities. In some cases, GM3S deficiency is associated with an ST3GAL5 c.862C>T founder variant segregating with a population-specific carrier frequency of ˜4%. GM3S mediates synthesis of GM3, which serves as the common precursor for all cerebral gangliosides. GM3S deficiency abolishes ganglioside biosynthesis. ST3GAL5 c.862C>T homozygotes appear healthy at birth, but develop progressive microcephaly, neurodevelopmental stagnation, intractable epilepsy, irritability, insomnia, deafness, blindness, and dyskinesia within a few months of life. No treatment is currently available.

### SUMMARY

Aspects of the disclosure relate to compositions and methods for expressing GM3 synthase (GM3S) proteins in a subject. The disclosure is based, in part, on expression constructs encoding one or more GM3S isoforms (e.g., one or more Ia Type 1 isoforms, Ia Type 2 isoforms, 1b Type 1 isoforms, Ib Type 2 isoforms, Ic isoforms, and combinations thereof). In some embodiments, expression constructs described by the disclosure are useful for treating diseases associated with GM3S deficiency.

Accordingly, in some aspects, the present disclosure provides a method for treating a GM3 synthase (GM3S) deficiency in a subject in need thereof, the method comprising administering to a subject having a GM3 synthase (GM3S) deficiency an effective amount of a recombinant adeno-associated virus (rAAV) via intracerebroventricular (ICV) administration, wherein the rAAV comprises: (i) a capsid protein; and (ii) an isolated nucleic acid comprises a transgene having a nucleic acid sequence encoding one or more monosialodihexosylganglioside synthase (GM3S) protein isoforms, and wherein the transgene is flanked by adeno-associated virus (AAV) inverted terminal repeats (ITRs).

In some aspects, the present disclosure provides a method for treating a GM3 synthase (GM3S) deficiency in a subject in need thereof, the method comprising administering to a subject having a GM3 synthase (GM3S) deficiency a composition comprising an effective amount of a recombinant adeno-associated virus (rAAV) and a pharmaceutically acceptable carrier via intracerebroventricular (ICV) administration, wherein the rAAV comprises: (i) a capsid protein; and (ii) an isolated nucleic acid comprises a transgene having a nucleic acid sequence encoding one or more monosialodihexosylganglioside synthase (GM3S) protein isoforms, and wherein the transgene is flanked by adeno-associated virus (AAV) inverted terminal repeats (ITRs).

In some embodiments, the transgene encodes a one or more GM3S Ia isoforms. In some embodiments, the one or more GM3S isoforms are GM3S Ia Type 1 isoforms. In some embodiments, the one or more GM3S Ia Type 1 isoforms are translated from an M2 initiation codon. In some embodiments, the one or more GM3S Ia Type 1 isoforms are translated from an M3 initiation codon. In some embodiments, the one or more GM3S isoforms are GM3S Ia Type 2 isoforms. In some embodiments, the one or more GM3S Ia Type 2 isoforms are translated from an M1 initiation codon. In some embodiments, the one or more GM3S Ia Type 2 isoforms are translated from an M2 initiation codon. In some embodiments, the one or more GM3S Ia Type 2 isoforms are translated from an M3 initiation codon.

In some embodiments, the transgene encodes one or more GM3S Ib isoforms. In some embodiments, the one or more GM3S isoforms are GM3S Ib Type 1 isoforms. In some embodiments, the one or more GM3S Ib Type 1 isoforms are translated from an M2 initiation codon. In some embodiments, the one or more GM3S Ib Type 1 isoforms are translated from an M3 initiation codon.

In some embodiments, the one or more GM3S isoforms are GM3S Ib Type 2 isoforms. In some embodiments, the one or more GM3S Ib Type 2 isoforms are translated from an M2 initiation codon. In some embodiments, the one or more GM3S Ib Type 2 isoforms are translated from an M3 initiation codon.

In some embodiments, the transgene encodes one or more GM3S Ic isoforms. In some embodiments, the one or more GM3S Ic isoforms are translated from an M2′ initiation codon.

In some embodiments, the one or more GM3S Ic isoforms are translated from an M2 initiation codon. In some embodiments, the one or more GM3S Ic isoforms are translated from an M3 initiation codon.

In some embodiments, the transgene further comprises a ST3GAL5 5′ untranslated region (5′UTR). In some embodiments, the transgene does not include a ST3GAL5 5′UTR.

In some embodiments, the transgene further comprises a Kozak sequence (GCCACC) operably linked to the nucleic acid sequence encoding the one or more GM3S protein isoforms.

In some embodiments, the each of the one or more GM3S protein isoforms comprises or consists of the amino acid sequence set forth in any one of SEQ ID NOs: 8-10.

In some embodiments, the transgene further comprises a promoter operably linked to the nucleic acid sequence encoding the one or more GM3 synthase protein isoforms. In some embodiments, the promoter is a chicken beta-actin (CBA) promoter, optionally wherein the CBA promoter comprises a CMV enhancer sequence. In some embodiments, the promoter is a hST3GAL5 promoter. In some embodiments, the promoter is a truncated hST3GAL5 promoter. In some embodiments, the promoter is a neuron-specific promoter. In some embodiments, the neuron-specific promoter is a Synapsin I (Syn1) promoter.

In some embodiments, the AAV ITR is an AAV2 ITR or a variant thereof.

In some embodiments, the nucleic acid sequence encoding the one or more GM3S protein isoforms is codon-optimized. In some embodiments, the isolated nucleic acid comprising the sequence set forth in any one of SEQ ID NOs: 1-7, and 11-43.

In some embodiments, the isolated nucleic acid further comprising one or more miRNA binding sites. In some embodiments, the one or more miRNA binding sites are miR-122 binding sites. In some embodiments, the one or more miRNA binding sites are positioned between the last codon of the nucleic acid sequence encoding the one or more GM3S protein isoforms and a polyA tail region.

In some embodiments, the capsid protein is an AAV9 capsid protein or a variant thereof. In some embodiments, the AAV9 capsid protein variant is AAV.PHP.B capsid protein, AAV.PHP.eb capsid protein, or AAV Cap-Mac capsid protein. In some embodiments, the rAAV is formulated for delivery to the brain.

In some embodiments, the rAAV described herein is a single-stranded rAAV. In some embodiments, the rAAV described herein is a self-complementary rAAV.

In some embodiments, the subject is characterized as having one or more mutations in a ST3GAL5 gene. In some embodiments, the one or more mutations occurs at position c.862, optionally wherein the mutation is C862T. In some embodiments, the one or more mutations occurs at position c.1063, optionally wherein the mutation is G1063A. In some embodiments, the one or more mutations occurs at positions c.584 and c.601, optionally wherein the mutations are G584C and G601A.

In some embodiments, the method further comprising administering miR-122 to the subject.

In some embodiments, the administration via ICV reduces acute liver toxicity relative to administration via a systemic route in a subject.

In some aspects, the present disclosure also provides a recombinant adeno associated virus (rAAV) comprising: (i) an AAV Cap-Mac capsid protein; and (ii) an isolated nucleic acid comprising a transgene having a nucleic acid sequence encoding one or more monosialodihexosylganglioside synthase (GM3S) Ia Type 2 protein isoforms, and wherein the transgene is flanked by adeno-associated virus (AAV) inverted terminal repeats (ITRs).

### BRIEF DESCRIPTION OF DRAWINGS

FIGS. 1A-1C show human ST3GAL5 protein isoforms and constructs expressing hST3GAL5 and their expression in Hela cells. FIG. 1A shows a schematic of a hST3GAL5-1a-2 RNA variant has at least three protein products that initiate from different start codons. FIG. 1B shows seven AAV constructs expressing the human ST3GAL5 cDNA, driven by the CMV/CB promoter. FIG. 1C shows a Western blot showing hST3GAL5 expression following transfection in Hela cells with the corresponding plasmids. All the plasmids can produce the desired isoforms and the 5′-UTR decreases the protein expression in Hela cells.

FIGS. 2A-2C show constructs expressing hST3GAL5 protein in patient-derived fibroblast cells. FIG. 2A shows patient-derived fibroblast cells genotype information. FIG. 2B shows a Western blot showing hST3GAL5 expression following transfection in fibroblast cells with the corresponding plasmids. All the plasmids produce the desired isoforms. FIG. 2C shows anti-GM3 immunofluorescence analysis in the fibroblast cells. ST3GAL5+/M is the healthy control line. ST3GAL5 M/M is generated from patient. ST3GAL5 M/M either remains untreated, or treated with lentivirus.

FIGS. 3A-3B show patient iPSC-induced cortical neurons. FIG. 3A shows a flow chart showing the cortical neuron generation timeline. FIG. 3B shows immunofluorescence staining showing the cortical neuron markers from D44 induced cortical neurons.

FIG. 4 shows anti-brain gangliosides analysis in iPSC-induced cortical neurons. The figure includes DAPI stain for nucleus, and GD1a, GD1b, or GT1b staining from top row to bottom row. ST3GAL5+/+ is the healthy control line. ST3GAL5 M/M is generated from patient iPSC. ST3GAL5 M/M either remains untreated, or treated with lentivirus.

FIGS. 5A-5E show intracerebroventricular (ICV) administration restored gangliosides production and improves animal growth. FIG. 5A shows ICV administration to wildtype neonatal mice induced robust transgene expression. FIG. 5B shows anti-gangliosides immunofluorescence analysis in St3gal5 knock-out animal brain. FIG. 5C shows mass spectrum analysis in St3gal5 knock-out animal brain. FIG. 5D shows ICV administration to St3gal5^{-/-} B4galnt1 double knock-out mice extended the median survival time. FIG. 5E shows ICV administration to St3gal5^{-/-} B4galnt1 double knock-out mice improved their physical growth.

FIGS. 6A-6B show quantification of in vivo rAAV genome delivery and transgene expression of wild-type mice by droplet digital PCR (ddPCR). FIG. 6A shows rAAV genome delivery data. FIG. 6B shows transgene expression data. Each dot represents the tissue from one animal (n=3-5).

FIGS. 7A-7C show quantification of in vivo rAAV genome delivery and transgene expression of wild-type mice by droplet digital PCR (ddPCR) and Western blot. FIG. 7A shows rAAV genome delivery by ddPCR. FIGS. 7B-7C show transgene expression by ddPCR and Western blot. Each dot represents the tissue from one animal (n=3-5).

FIGS. 8A-8B are tables showing the animal survival post AAV9_hST3GAL5 vectors injection at multiple doses. FIG. 8A is a table showing neonatal facial vein injection of the rAAV listed therein. FIG. 8B is a table showing young adult tail vein injection of the rAAV listed therein.

FIG. 9 shows liver morphology showing the acute liver damage at 2-days post injection. Panel a shows TUNEL staining on AAV9-hST3GAL5 systemic injection. Panel b shows TUNEL staining on PBS systemic injection. Panel c shows H&E staining on AAV9-hST3GAL5 systemic injection. Panel d shows H&E staining on PBS systemic injection.

FIG. 10 shows the ALT level of wildtype young adult C57/BL6 mice post AAV9_hST3GAL5 vector I.V. injection. Blood are taken from animals weekly. Each dot represents the tissue from one animal (n=3-5).

FIG. 11 shows St3gal5 expression in liver at 2-days post systemic injection.

FIG. 12 shows examples of constructs expressing hST3GAL5 under different expression cassettes. Cartoons showing four AAV expression cassettes expressing the human ST3GAL5 cDNA, driven by hSyn1 promoter, hST3GAL5 promoter and miR122 binding sites, hST3GAL5 endogenous full-length promoter, and hST3GAL5 endogenous truncated promoter.

FIG. 13A-13B show miR122 binding sites reduced the liver toxicity in young adult C57/BL6 animal but did not rescue the neonatal toxicity. FIG. 13A shows neonatal facial vein injection of miR122 binding sites vectors did not improve animal survive. FIG. 13B shows young adult tail vein injection of miR122 binding sites vectors reduced the liver ALT level. Each dot represents the tissue from one animal (n=1-3).

FIG. 14 shows miR-122 expression differences among neonatal liver, adult liver, and heart samples. Liver from 0-day, 1-day, and 6-week-old wildtype mice, and heart from 6-week-old wildtype mice were collected.

FIG. 15 shows the combination of Syn1 promoter and miR122 binding sites and human ST3GAL5 endogenous promoter alleviated the liver toxicity. TLR2, TLR9, and downstream cytokines (TNFa, IL6, IL12b) mRNA levels from AAV9 vectors injected liver were detected by ddPCR, respectively.

FIG. 16A-16D show that GM3 synthase deficiency is caused by loss-of-function mutation of ST3GAL5. FIG. 16A is a schematic showing de novo gangliosides synthesis pathway. ST3GAL5 uses lactosylceramides (LacCer) as substrate to synthesize GM3, the precursor of all other gangliosides. B4GALNT1 is another key enzyme to catalyze the complex gangliosides formation. Loss-of-function mutations in ST3GAL5 and B4GALNT1 cause GM3 synthase deficiency (GM3SD) and Hereditary Spastic Paraplegia Type 26 (HSP26), respectively. FIG. 16B is a schematic of human ST3GAL5 DNA genome and the most abundant mRNA isoform noted in NCBI (NM_003896). M1, M2, and M3 represent three initiating starting codon methionine. Stop codon TGA is at exon 10 and Amish mutation (p.862C>T) locates at exon 9. cDNA initiating from M1 (ORF1), M2 (ORF2), M3 (ORF3), and Kozak+M3 (KORF3) possesses 1257 bp, 1173 bp, 1089 bp, and 1095 bp size, respectively. Black boxes, exons; black lines, introns; dashed black lines, spliced introns. FIG. 16C shows a construct expressing ubiquitous human ST3GAL5 ORF is shown. FIG. 16D is a representative Western blot images of ST3GAL5 protein expression via different ORF transfection in HeLa cell.

FIGS. 17A-17C show that ST3GAL5 replacement restores gangliosides production in cortical neurons. FIG. 17A shows the workflow to examine restoration of gangliosides production in patient induced pluripotent stem cell (iPSC) differentiated cortical neurons by lentiviral vectors expressing ST3GAL5 ORFs. FIG. 17B shows representative images of neuronal markers in ST3GAL5^{+/+} and ST3GAL5^{mut/mut} iPSC differentiated cortical neurons. Neuron-specific class III beta-tubulin (Tuj1), COUP-TF-interacting protein 2 (Ctip2), T-Box Brain Transcription Factor 1 (TBR1), and nuclei were evaluated. +/+, wildtype; m/m, St3gal5^{mut/mut}. FIG. 17C shows representative images of major brain gangliosides in cortical neurons by lentiviral vectors expressing ST3GAL5 ORFs. GD1a, GD1b, GT1b, and nuclei were evaluated. +/+, wildtype; m/m, St3gal5′^{mut/mut}.

FIGS. 18A-18D show I.C.V. delivery of ST3GAL5 restores gangliosides production in St3 gal5^{-/-} mouse model. FIG. 18A shows a schematic of intracerebroventricular (I.C.V.) delivery of ubiquitous human ST3GAL5 cDNA Kozak methionine 3 isoform (KM3) in St3gal5^{-/-} mouse model. FIG. 18B shows droplet digital PCR (ddPCR) quantification of rAAV9 genome and human ST3GAL5 transgene in the brain (Br), liver (Li), and heart (He) of rAAV9.CB.hST3GAL5 treated St3gal5^{+/+} mice. Mouse endogenous St3 gal5 mRNA was quantified from brain (Br), liver (Li), and heart (He) of St3gal5^{+/+} mice. Data are mean±s.d. of 7-10 animals per group. Statistical analysis was performed by t-test. **p<0.01. FIG. 18C shows mass spectrometry (MS) quantification of GM3 (18:0), GM2 (18:0), LacCer (18:0), and GM1 (18:0) from the brain of St3gal5^{+/+} and St3 gal5^{-/-} mice, with (+) or without (−) rAAV9.CB.hST3GAL5 treatment. Data are mean±s.d. of 3 animals per group. Statistical analysis was performed by one-way ANOVA, followed by Sidak's multiple comparisons test. *p<0.05. **p<0.01. ***p<0.001. ****p<0.0001, ns: not significant. FIG. 18D shows representative images of major brain gangliosides in cortex of St3gal5^{+/+} and St3gal5^{-/-} mice, with (+) or without (−) rAAV9.CB.hST3GAL5 treatment. GD1a, GD1b, and nuclei are evaluated. Quantification is in FIG. 25.

FIGS. 19A-19G show liver de-targeting eliminates ST3GAL5 overexpression induced toxicity. FIG. 19A shows median survival of post AAV9.CB.hST3GAL5 intravenous (I.V.) delivery. Data are plotted as probability of survival from 4-11 animals. FIG. 19B shows a schematic of facial vein delivery of AAV9.CB.hST3GAL5, or AAV9. EGFP, or AAV9.empty, or AAV9.Syn1.hST3GAL5.miR122BS, or PBS in wildtype mice. FIG. 19C shows ddPCR quantification of human ST3GAL5 cDNA in the liver of wildtype mice with rAAV9.CB.hST3GAL5 or rAAV9.hSyn1.hST3GAL5.miR122BS treatments and endogenous mouse St3 gal5 from PBS treatment. Data are mean±s.d. of 3-4 animals per group. Statistical analysis was performed by Student t-test. *p<0.05. **p<0.01. FIG. 19D shows volcano plots showing differential expressed genes in mouse livers. Adjusted P value <=0.05, foldchange >=2.

FIG. 19E shows a graph depicting significantly enriched pathways for differential expressed genes between liver from wildtype mice injected with PBS and rAAV9.CB.hST3GAL5 using Gene Set enrichment analysis (GSEA). FIG. 19F shows representative images of hematoxylin and eosin (H&E) staining of liver sections from wildtype mice injected with rAAV9.CB.hST3GAL5 or PBS or rAAV9.hSyn1.hST3GAL5.miR122BS. FIG. 19G show representative images of terminal deoxynucleotidyl transferase dUTP nick end labeling (TUNEL) staining of liver sections from wildtype mice injected with rAAV9.CB.hST3GAL5 or PBS or rAAV9.hSyn1.hST3GAL5.miR122BS.

FIGS. 20A-20D show second generation of ST3GAL5 replacement vector restores gangliosides production in St3 gal5^{-/-} mouse model. FIG. 20A show a schematic of I.C.V. delivery of neuron-specific human ST3GAL5 cDNA KM3 in St3gal5^{-/-} mouse model. FIG. 20B show ddPCR quantification of rAAV9 genome and human ST3GAL5 cDNA in the cortex, hippocampus (Hippo), liver and heart of ssAAV9.hSyn1.hST3GAL5.miR122BS or scAAV9.hSyn1.hST3GAL5.miR122BS treated St3gal5^{-/-} mice. Data are mean±s.d. of 4-9 animals per group. Statistical analysis was performed by student t-test. *p<0.05. **p<0.01, ns: not significant. FIG. 20C shows mass spectrometry (MS) quantification of GM3 (18:0), GM2 (18:0), and LacCer (18:0) from the brain of St3gal5^{+/+} and St3 gal5−/− mice, with ssAAV9.hSyn1.hST3GAL5.miR122BS or scAAV9.hSyn1.hST3GAL5.miR122BS or no treatment. Data are mean±s.d. of 3-4 animals per group. Statistical analysis was performed by one-way ANOVA, followed by Sidak's multiple comparisons test. *p<0.05, **p<0.01, ***p<0.001, ns: not significant. FIG. 20D shows representative images of major brain gangliosides in cortex of St3gal5^{+/+} and St3gal5^{-/-} mice, with ssAAV9.hSyn1.hST3GAL5.miR122BS or scAAV9.hSyn1.hST3GAL5.miR122BS or no treatment. GD1a, GD1b, and nuclei were evaluated. Quantification is in FIG. 25.

FIGS. 21A-21H show second generation of ST3GAL5 replacement vector rescues phenotypical changes in St3gal5^{+/+}/B4galnt14 mouse model. FIG. 21A shows a schematic of I.C.V. delivery of neuron-specific human ST3GAL5 cDNA KM3 in St3gal5^{-/-}/B4galnt1^{-/-} mouse model. FIG. 21B shows median survival of St3gal5^{-/-}/B4galnt1^{-/-} mice, with ssAAV9.hSyn1.hST3GAL5.miR122BS or scAAV9.hSyn1.hST3GAL5.miR122BS or no treatments. Data are plotted as probability of survival from 7-20 animals. Statistical analysis was performed by Lon-rank (Mantel-Cox) test. ****p<0.0001. FIG. 21C shows a time course body weight from postnatal pups aging from 3 days old to 21 days old. Data are mean±s.d. of 10 animals. Statistical analysis was performed by two-way ANOVA, followed by Sidak's multiple comparisons test. *p<0.05, **p<0.01. FIG. 21D shows body weight at postweaning stage. Data are represented as mean±s.d. of 5-8 animals. FIG. 21E shows negative geotaxis successful rate from postnatal pups aging from 9 days old to 15 days old. Data are normalized from 10 animals. FIG. 21F shows quantification of rotarod assay for St3gal5^{+/-}/B4galnt1^{-/-} mice and ssAAV9.hSyn1.hST3GAL5.miR122BS or scAAV9.hSyn1.hST3GAL5.miR122BS treated St3 gal5^{-/-}/B4galnt1^{-/-} mice at 6 weeks old or 10 weeks old. Data are represented as mean±s.d. of 4-7 animals. Statistical analysis was performed by one-way ANOVA, followed by Sidak's multiple comparisons test. ns: not significant, *p<0.05. **p<0.01. FIG. 21G shows quantification of brain weight from wildtype mice or St3gal5^{-/-}/B4galnt1^{-/-} mice, with ssAAV9.hSyn1.hST3GAL5.miR122BS or scAAV9.hSyn1.hST3GAL5.miR122BS or no treatments at 3 weeks old. Data are mean±s.d. of 4-8 animals. Statistical analysis was performed by one-way ANOVA, followed by Sidak's multiple comparisons test. *p<0.05, **p<0.01. FIG. 21H shows images of mouse hindlimb clasping from St3gal5^{-/-}/B4galnt1^{-/-} mouse with scAAV9.hSyn1.hST3GAL5.miR122BS treatment or St3gal5^{+/-}/B4galnt1^{-/-} mouse.

FIGS. 22A-22C show second generation of ST3GAL5 replacement vector rescues brain histology in St3gal5^{-/-}/B4galnt1^{-/-} mouse model. FIG. 22A show representative images of hematoxylin and eosin (H&E) staining of cerebellum sections from wildtype mice and St3gal5^{-/-}/B4galnt1^{-/-} mice with ssAAV9.hSyn1.hST3GAL5.miR122BS or scAAV9.hSyn1.hST3GAL5.miR122BS or no treatments. Black rectangle: zoom in area. FIG. 22B show representative images of terminal deoxynucleotidyl transferase dUTP nick end labeling (TUNEL) staining of brain sections (cortex, hippocampus, cerebellum) from wildtype mice and St3gal5^{-/-}/B4galnt1^{-/-} mice with ssAAV9.hSyn1.hST3GAL5.miR122BS or scAAV9.hSyn1.hST3GAL5.miR122BS or no treatments. FIG. 22C shows representative images and quantification of anti-GFAP immunostaining of brain sections (cortex, hippocampus, midbrain) from wildtype mice and St3gal5^{-/-}/B4galnt1^{-/-} mice with ssAAV9.hSyn1.hST3GAL5.miR122BS or scAAV9.hSyn1.hST3GAL5.miR122BS or no treatments. Mean intensity was quantified by Fiji. Data are represented as mean±s.d. of 3-5 animals. Statistical analysis was performed by one-way ANOVA, followed by Sidak's multiple comparisons test.

FIGS. 23A-23G show co-delivery of ST3GAL5 and B4GALNT1 vectors normalize St3 gal5^{-/-}/B4galnt1^{-/-} mouse model. FIG. 23A shows schematic of I.C.V. co-delivery of AAV vectors expressing ST3GAL5 and B4GALNT1 cDNA respectively in St3gal5^{-/-}/B4galnt1^{-/-} mouse model. FIG. 23B shows median survival of St3gal5^{-/-}/B4galnt1^{-/-} mice, with or without co-delivery of ST3GAL5 and B4GALNT1. Data are plotted as probability of survival from 8-20 animals. Statistical analysis was performed by Lon-rank (Mantel-Cox) test. ****p<0.0001. FIG. 23C shows a time course body weight from postnatal pups aging from 3 days old to 21 days old. Data are mean±s.d. of 10 animals. Statistical analysis was performed by two-way ANOVA, followed by Sidak's multiple comparisons test. *p<0.05, **p<0.01, ns: not significant. FIG. 23D shows body weight of male and female at postweaning stage. Data are represented as mean±s.d. of 3-5 animals. FIG. 23E shows negative geotaxis successful rate from postnatal pups aging from 9 days old to 15 days old. Data are normalized from 7-9 animals. FIG. 23F shows quantification of rotarod assay for St3gal5^{+/-}/B4galnt1^{-/-} mice and ssAAV9.hSyn1.hST3GAL5.miR122BS or scAAV9.hSyn1.hST3GAL5.miR122BS or dual vectors treated St3 gal5^{-/-}/B4galnt1^{-/-} mice at 6 weeks old and 10 weeks old. Data are represented as mean±s.d. of 5-8 animals. FIG. 23G shows images of mouse hindlimb from St3gal5^{-/-}/B4galnt1^{-/-} mouse with dual vectors treatment or St3 gal5^{+/-}/B4galnt1^{-/-} mouse.

FIGS. 24A-24B show that St3gal5^{-/-} B4galnt1^{-/-} mice exhibit impaired growth and motor function. FIG. 24A shows a time course body weight from postnatal pups aging from 3 days old to 21 days old. Data are mean±s.d. of 8-10 animals. Statistical analysis was performed by two-way ANOVA, followed by Sidak's multiple comparisons test. ***P<0.001, ****P<0.0001, ns: not significant. FIG. 24B shows negative geotaxis successful rate from postnatal pups aging from 9 days old to 15 days old. Data are normalized from 10 animals.

FIG. 25 shows quantification of gangliosides GD1a and GD1b. Quantification of mean intensity of GD1a or GD1b immunofluorescence from cortex of St3gal5+/+ and St3gal5−/− mice, with (+) or without (−) AAV.ST3GAL5 treatments. ROA, route of administration. Data are mean±s.d. of 3-4 animals per group. Statistical analysis was performed by one-way ANOVA, followed by Sidak's multiple comparisons test. *p<0.05, **p<0.01, ns: not significant.

FIG. 26 shows that I.C.V. delivery of ST3GAL5 restores limited peripheral gangliosides production in St3gal5/mouse model. Mass Spectrometry (MS) quantification of GM3 (16:0, 18:0), GM2 (16:0, 18:0), and LacCer (16:0, 18:0) from serum of St3gal5^{+/+} and St3 gal5^{-/-} mice, with (+) or without (−) rAAV9.CB.hST3GAL5 treatment. Data are mean±s.d. of 3 animals per group. Statistical analysis was performed by one-way ANOVA, followed by Sidak's multiple comparisons test. *p<0.05. **p<0.01, ns: not significant.

FIGS. 27A-27F show I.C.V. delivery of ST3GAL5 rescues phenotypical changes in St3 gal5^{-/-}/B4galnt1^{-/-} mouse model. FIG. 27A shows a schematic of intracerebroventricular (I.C.V.) delivery of ubiquitous human ST3GAL5 cDNA Kozak methionine 3 isoform (KM3) in St3 gal5^{-/-}/B4galnt1^{-/-} mouse model. FIG. 27B shows median survival of St3gal5^{-/-}/B4galnt1^{-/-} mice, with or without rAAV9.CB.hST3GAL5 treatment. Data are plotted as probability of survival from 7-17 animals. Statistical analysis was performed by Lon-rank (Mantel-Cox) test. ***p<0.001. FIG. 27C shows a time course body weight from postnatal pups aging from 3 days old to 21 days old. Data are mean±s.d. of 8-10 animals. Statistical analysis was performed by two-way ANOVA, followed by Sidak's multiple comparisons test. *p<0.05, **p<0.01. FIG. 27D shows negative geotaxis successful rate from postnatal pups aging from 9 days old to 15 days old. Data are normalized from 8-10 animals. FIG. 27E shows body weight of male and female at postweaning stage. Data are represented as mean±s.d. of 3-4 animals. FIG. 27F shows quantification of rotarod assay for St3gal5^{+/-}/B4galnt1^{-/-} mice and rAAV9.CB.hST3GAL5 treated St3 gal5^{-/-}/B4galnt1^{-/-} mice at 6 weeks old. Data are represented as mean±s.d. of 3-5 animals. Statistical analysis was performed by student t-test. **p<0.01.

FIGS. 28A-28B show ST3GAL5 overexpression in liver induces cellular toxicity. FIG. 28A shows qPCR quantification of Chop and Tnfa mRNA in the liver of wildtype mice with rAAV9.CB.hST3GAL5 or rAAV9.hSyn1.hST3GAL5.miR122BS or PBS treatments. Data are mean±s.d. of 4 animals per group. Statistical analysis was performed by one-way ANOVA, followed by Sidak's multiple comparisons test. *p<0.05, **p<0.01, ns: not significant. FIG. 28B shows quantification of enzyme-linked immunosorbent assay (ELISA) of TNFa, IL1a, CCL2, and CCL3 protein in the liver of wildtype mice with rAAV9.CB.hST3GAL5 or rAAV9.hSyn1.hST3GAL5.miR122BS or PBS treatments. Data are mean±s.d. of 4 animals per group. Statistical analysis was performed by one-way ANOVA, followed by Sidak's multiple comparisons test. *p<0.05, **p<0.01. ***p<0.001, ns: not significant.

FIG. 29 shows packaging efficiency of CB.hST3GAL5 and Syn1.hST3GAL5.miR122BS. Quantification of vector titer from AAV9 vector preparation. Data are mean of 3-6 preparations per group.

FIG. 30 shows that second generation of ST3GAL5 replacement vector dose not restore peripheral gangliosides production in St3gal5^{-/-} mouse model. Mass Spectrometry (MS) quantification of GM3 (16:0, 18:0), GM2 (16:0, 18:0), and LacCer (16:0, 18:0) from serum of St3 gal5^{+/+} and St3 gal5^{-/-} mice, with ssAAV9.hSyn1.hST3GAL5.miR122BS or scAAV9.hSyn1.hST3GAL5.miR122BS or no treatment. Data are mean±s.d. of 3-4 animals per group. Statistical analysis was performed by one-way ANOVA, followed by Sidak's multiple comparisons test. *p<0.05. **p<0.01. ***p<0.001. ****p<0.0001, ns: not significant.

FIGS. 31A-31F show that systematic delivery of second generation ST3GAL5 vector via AAV PHP.eB rescues GM3SD mouse models. FIG. 31A shows a schematic of intravenous (I.V.) delivery of neuron-specific human ST3GAL5 cDNA KM3 in mouse models. FIG. 31B shows a time course body weight from postnatal pups aging from 3 days old to 21 days old. Data are mean±s.d. of 7-10 animals. FIG. 31C shows negative geotaxis successful rate from postnatal pups aging from 9 days old to 15 days old. Data are normalized from 8-10 animals.

FIG. 31D shows median survival of St3gal5^{-/-}/B4galnt1^{-/-} mice, with scAAV9.hSyn1.hST3GAL5.miR122BS or PHP.eB.hSyn1.hST3GAL5.miR122BS or no treatment. Data are plotted as probability of survival from 7-20 animals. Statistical analysis was performed by Lon-rank (Mantel-Cox) test. ***p<0.001. FIG. 31E shows ddPCR quantification of AAV genome and human ST3GAL5 cDNA in the cortex, hippocampus (Hippo), liver and heart of scAAV9.hSyn1.hST3GAL5.miR122BS or PHP.eB.hSyn1.hST3GAL5.miR122BS treated St3gal5^{-/-} mice. Data are mean±s.d. of 2-8 animals per group. FIG. 31F shows representative images of major brain gangliosides in cortex of St3gal52^{-/-} mice with scAAV9.hSyn1.hST3GAL5.miR122BS or PHP.eB.hSyn1.hST3GAL5.miR122BS treatment. GD1a, GD1b and nuclei were evaluated. Quantification is in FIG. 25.

### DETAILED DESCRIPTION

In some aspects, the disclosure relates to compositions and methods for expressing a transgene encoding one or more GM3 synthase (GM3S) proteins in a cell or subject. In some embodiments, the transgene encodes an isolated nucleic acid. In some embodiments, the isolated nucleic acid is comprised in a recombinant adeno-associated virus (rAAV). In some aspects, the disclosure relates to methods for treating GM3 synthase deficiency in a subject in need thereof by administering an rAAV expressing one or more Ganglioside GM3 synthase (GM3S) isoforms via intracerebroventricular (ICV) administration.

Methods and compositions described by the disclosure may be utilized, in some embodiments, to treat diseases and disorders associated with GM3S deficiency.

### Isolated Nucleic Acids

A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

Aspects of the disclosure relate to isolated nucleic acids encoding one or more GM3S proteins. GM3 synthase (GM3S), also referred to as Lactosylceramide alpha-2,3-sialyltransferase and SATI, is an enzyme that catalyzes formation of ganglioside GM3 using a lactosylceramide substrate. In some embodiments, a human GM3S protein is encoded by an mRNA transcript having the sequence set forth in any one of NCBI Reference Sequence Accession Numbers: NM_001042437, NM_003896, NM_001354226, NM_001354227, and NM_001354233. In some embodiments, a human GM3S protein comprises the amino acid sequence set forth in any one of NCBI Reference Sequence Accession Numbers: NP_001035902, NP_003887, NP_001341155, NP_001341156, and NP_001341162.

Human GM3S transcripts (e.g., GM3S proteins translated from such transcripts) are generally classified into three isoforms, GM3S-Ia, -Ib, and -Ic, according to the position of transcription initiation, exon 1, 2, or 4. Additionally, GM3S-Ia and -Ib isoforms are each further classified into two types: GM3S-Ia Type 1 and Type 2 variants, and GM3S-Ib Type 1 and Type 2 variants, respectively, reflecting alternative splicing of exon 3. The structural features of GM3 isoforms are known, for example as described in Uemura et al. Mol Biol Cell. 20(13):3088-3100.

In some embodiments, an isolated nucleic acid includes an RNA transcript having one or more initiation codons (referred to as M1, M2, M3, etc.) which encodes one or more variants of a GM3S isoform (e.g., one or more variants of a Ia Type 1 isoform, one or more variants of a Ia Type 2 isoform, one or more variants of a Ib Type 1 isoform, one or more variants of a Ib Type 2 isoform, etc.). An initiation codon may be a codon encoding a methionine (M, encoded by AUG codon) or a codon encoding an amino acid that is not methionine but is capable of initiation of protein translation (e.g., leucine, encoded by CUG codon). In some embodiments, an initiation codon consists of the nucleotide sequence AUG. In some embodiments, the isolated nucleic acids described herein encodes one or more variants of a GM3S Ia Type 2 isoform (e.g., GM3S Ia Type 2 M1, GM3S Ia Type 2 M2, or GM3S Ia Type 2 M3 variants). In some embodiments, a human GM3S Ia Type 2 isoform (e.g., GM3S Ia Type 2 M1, GM3S Ia Type 2 M2, or GM3S Ia Type 2 M3 variants) comprises an amino acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOs: 8-10.

The position of the one or more initiation codons within an isolated nucleic acid may vary. In some embodiments, an initiation codon is positioned at the N-terminus of a nucleic acid sequence encoding one or more GM3S proteins (e.g., the first three nucleotides of the nucleic acid encode an AUG codon). In some embodiments, an initiation codon is positioned between 10 nucleotide bases and 50 nucleotide bases downstream from the N-terminus of a nucleic acid sequence encoding one or more GM3S proteins. In some embodiments, an initiation codon is positioned between 20 nucleotide bases and 100 nucleotide bases downstream from the N-terminus of a nucleic acid sequence encoding one or more GM3S proteins. In some embodiments, an initiation codon is positioned between 50 nucleotide bases and 500 nucleotide bases downstream from the N-terminus of a nucleic acid sequence encoding one or more GM3S proteins. In some embodiments, an initiation codon encodes a methionine (M) positioned between 1 amino acid and 30 amino acids downstream from the N-terminus of a nucleic acid sequence encoding one or more GM3S proteins. In some embodiments, an initiation codon encodes a methionine (M) positioned between 10 amino acid and 100 amino acids downstream from the N-terminus of a nucleic acid sequence encoding one or more GM3S proteins.

The disclosure is based, in part, on expression constructs encoding one or more GM3S isoforms or variants (e.g., one or more Ia Type 1 isoforms, Ia Type 2 isoforms, 1b Type 1 isoforms, Ib Type 2 isoforms, Ic isoforms, and combinations thereof). In some embodiments, an isolated nucleic acid encodes one or more GM3S Ia Type 1 variants (e.g., M1 isoform, M2 isoform, M3 isoform, or any combination of the foregoing.) In some embodiments, an isolated nucleic acid encodes one or more GM3S Ia Type 2 variants (e.g., M1 isoform, M2 isoform, M3 isoform, or any combination of the foregoing). In some embodiments, an isolated nucleic acid encodes one or more GM3S Ib Type 1 variants (e.g., M2 isoform, M3 isoform, or a combination thereof). In some embodiments, an isolated nucleic acid encodes one or more GM3S Ib Type 2 variants (e.g., M2 isoform, M3 isoform, or a combination thereof). In some embodiments, an isolated nucleic acid encodes one or more GM3S Ic Type 1 variants (e.g., M2′ isoform, M2 isoform, M3 isoform, or any combination of the foregoing).

In some aspects, the disclosure relates to isolated nucleic acids comprising or lacking certain regulatory sequences. In some embodiments, isolated nucleic acids and rAAVs described herein comprise (or lack) one or more of the following structural features (e.g., control or regulatory sequences): a 5′ untranslated region (5′UTR), a promoter, an intron, a Kozak sequence, one or more miRNA binding sites, a rabbit beta-globulin (RBG) poly A sequence, and a 3′ untranslated region (3′UTR). In some embodiments, one or more of the foregoing control sequences is operably linked to a nucleic acid sequence encoding one or more GM3S proteins.

The disclosure is based, in part, on isolated nucleic acids encoding one or more GM3S proteins (e.g., one or more GM3S isoforms or variants, for example GM3S Ia Type 2 M1, M2, and M3 variants, etc.) that lack a 5′ UTR (e.g., a ST5GAL5 5′ UTR) and/or lack a Kozak sequence (e.g., GCCACC). Without wishing to be bound by any particular theory, constructs lacking a 5′ UTR and/or a Kozak sequence express increased levels of multiple isoforms of GM3S proteins relative to expression constructs which include a 5′ UTR and/or a Kozak sequence.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame.

In some embodiments, a transgene comprises a nucleic acid sequence encoding one or more GM3S proteins operably linked to a promoter. A “promoter” refers to a DNA sequence recognized by the synthetic machinery of the cell, or introduced synthetic machinery, required to initiate the specific transcription of a gene. The phrases “operatively linked.” “operatively positioned.” “under control” or “under transcriptional control” means that the promoter is in the correct location and orientation in relation to the nucleic acid to control RNA polymerase initiation and expression of the gene.

Generally, a promoter can be a constitutive promoter, inducible promoter, or a tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an RNA pol II promoter. In some embodiments, a promoter is an RNA pol III promoter, such as U6 or H1. In some embodiments, a promoter is an RNA pol II promoter.

Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene (e.g., ST3GAL5 promoter or hSyn1 promoter) will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression. In some embodiments, the promoter is a ST3GAL5 promoter (e.g., human ST3GAL5 promoter). In some embodiments, the promoter is a truncated ST3GAL5 promoter (e.g., truncated human ST3GAL5 promoter). In some embodiments, the promoter is a neuron specific promoter. In some embodiments, the neuron specific promoter is a Synapsin I (Syn1) promoter.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some aspects, the disclosure relates to isolated nucleic acids comprising a transgene encoding one or more GM3S proteins, and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding one or more GM3S proteins, and a poly A sequence.

In some embodiments, a transgene comprises one or more (e.g., 1, 2, 3, 4, 5, or more) miRNA binding sites that de-target expression of the one or more GM3S proteins from liver cells. For example, in some embodiments, a transgene comprises one or more miR-122 binding sites.

In some embodiments, a transgene comprises one or more (e.g., 1, 2, 3, 4, 5, or more) miRNA binding sites that de-target expression of the one or more GM3S proteins from immune cells (e.g., antigen presenting cells (APCs), such as macrophages, dendrites, etc.). Incorporation of miRNA binding sites for immune-associated miRNAs may de-target transgene expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune-associated miRNA” is an miRNA preferentially expressed in a cell of the immune system, such as an antigen presenting cell (APC). In some embodiments, an immune-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune-associated miRNA is expressed is a B cell, T cell, Killer T cell, Helper T cell, γδ T cell, dendritic cell, macrophage, monocyte, vascular endothelial cell, or other immune cells. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152.

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (Sec, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are AAV2 ITRs.

In some embodiments, the AAV vector comprises, from 5′ to 3′: (i) a 5′ AAV2 ITR, (ii) a hSyn1 promoter, (iii) an intron, (iv) a nucleic acid sequence encoding one or more monosialodihexosylganglioside synthase (GM3S) protein isoforms, (v) a poly A, and (vi) a 3′ AAV ITR.

In some embodiments, the AAV vector comprises, from 5′ to 3′: (i) a 5′ AAV2 ITR, (ii) a hSyn1 promoter. (iii) an intron, (iv) a nucleic acid sequence encoding one or more monosialodihexosylganglioside synthase (GM3S) protein isoforms, (v) one or more miR-122 binding site; (vi) a poly A, and (vii) a 3′ AAV ITR.

In some embodiments, the AAV vector comprises, from 5′ to 3′: (i) a 5′ AAV2 ITR, (ii) a hST3GAL5 promoter, (iii) an intron, (iv) a nucleic acid sequence encoding one or more monosialodihexosylganglioside synthase (GM3S) protein isoforms, (v) a poly A, and (vi) a 3′ AAV ITR.

In some embodiments, the AAV vector comprises, from 5′ to 3′: (i) a 5′ AAV2 ITR, (ii) a hST3GAL5 promoter, (iii) an intron, (iv) a nucleic acid sequence encoding one or more monosialodihexosylganglioside synthase (GM3S) protein isoforms, (v) one or more miR-122 binding site; (vi) a poly A, and (vii) a 3′ AAV ITR.

In some embodiments, the AAV vector comprises, from 5′ to 3′: (i) a 5′ AAV2 ITR, (ii) a truncated hST3GAL5 promoter, (iii) an intron, (iv) a nucleic acid sequence encoding one or more monosialodihexosylganglioside synthase (GM3S) protein isoforms, (v) a poly A, and (vi) a 3′ AAV ITR.

In some embodiments, the AAV vector comprises, from 5′ to 3′: (i) a 5′ AAV2 ITR, (ii) a truncated hST3GAL5 promoter, (iii) an intron, (iv) a nucleic acid sequence encoding one or more monosialodihexosylganglioside synthase (GM3S) protein isoforms, (v) one or more miR-122 binding site; (vi) a poly A, and (vii) a 3′ AAV ITR.

Recombinant Adeno-Associated Viruses (rAAVs) and Other Vectors

Aspects of the disclosure relate to vectors comprising an isolated nucleic acid encoding one or more GM3S proteins. As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s) (e.g., ocular tissues, neurons). The AAV capsid is an important element in determining these tissue-specific targeting capabilities (e.g., tissue tropism). Thus, an rAAV having a capsid appropriate for the tissue being targeted can be selected.

In some embodiments, rAAVs of the disclosure comprise a nucleotide sequence as set forth in any one of SEQ ID NOs: 1-7 and 11-43, or encode one or more GM3s proteins each having an amino acid sequence as set forth in any one of SEQ ID NOs: 8-10. In some embodiments, rAAVs of the disclosure comprise a nucleotide sequence that is 99% identical, 95% identical, 90% identical, 85% identical, 80% identical, 75% identical, 70% identical, 65% identical, 60% identical, 55% identical, or 50% identical to a nucleotide sequence as set forth in SEQ ID NOs: 1-7 and 11-43.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for CNS tissue (e.g., brain tissue, spinal tissue, etc.). In some embodiments, an AAV capsid protein targets neuronal cells. In some embodiments, an AAV capsid protein is capable of crossing the blood-brain barrier (BBB).

In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP.B. AAV.PHP.eB, and variants of any of the foregoing. In some embodiments, an AAV capsid protein is of a serotype derived from a non-human primate, for example AAVrh8 serotype. In some embodiments, the AAV capsid protein is AAV9 or a variant thereof. In some embodiments, the AAV9 capsid variant is AAV PHP.B or AAV PHP.cB. In some embodiments, the AAV9 capsid variant is AAV Cap-Mac, e.g., AAV Cap-Mac capsid protein as described by Chuapoco et al., Intravenous gene transfer throughout the brain of infant Old World primates using AAV, bioRxiv 2022.01.08.475342).

In some embodiments, the rAAV is a single stranded AAV (ssAAV). An ssAAV, as used herein, refers to a rAAV with the coding sequence and complementary sequence of the transgene expression cassette on separate strands and packaged in separate viral capsids. In some embodiments, the rAAV is a self-complementary AAV (scAAV). A scAAV, as used herein, refers to an rAAV with both the coding and complementary sequence of the transgene expression cassette are present on each plus-and minus-strand genome. The coding region of a scAAV was designed to form an intra-molecular double-stranded DNA template. Upon infection, rather than waiting for cell-mediated synthesis of the second strand, the two complementary halves of scAAV associate to form one double stranded DNA (dsDNA) unit that is ready for immediate replication and transcription. In some embodiments, an rAAV comprising an isolated nucleic acid encoding the M3 variant of any one of the ST3GAL5 isoforms (e.g., ST3GAL5 Ia Type 2 M3 variant) is a self-complementary (sc) rAAV.

In some embodiments, an rAAV vector or rAAV particle comprises a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a coding sequence encoding a transgene (e.g., one or more GM3S proteins). A “host cell” refers to any cell that harbors, or is capable of harboring, a substance of interest. Often a host cell is a mammalian cell. In some embodiments, a host cell is a neuron. In some embodiments, a host cell is a photoreceptor cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a neuron or a glial cell (e.g., astrocyte, oligodendrocyte, etc.).

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. Sec, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. Sec, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. Sec, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

### Methods

Methods for delivering a transgene (e.g., an isolated nucleic acid encoding one or more GM3S proteins) to a subject are provided by the disclosure. In some embodiments, a subject tis administered one or more (e.g., 1, 2, 3, 4, 5, 6, or more) rAAVs, each rAAV encoding a different transgene (e.g., each encoding a different GM3S protein). The methods typically involve administering to a subject an effective amount of an isolated nucleic acid encoding the transgene. In some embodiments, expression constructs described by the disclosure are useful for treating diseases associated by GM3S deficiency.

In some aspects, the disclosure provides a method for treating a GM3 synthase (GM3S) deficiency in a subject in need thereof, the method comprising administering to a subject having a GM3 synthase (GM3S) deficiency an effective amount of an isolated nucleic acid, vector, or rAAV as described herein. A subject may be any mammalian organism, for example a human, non-human primate, horse, pig, dog, cat rodent, etc. In some embodiments a subject is a human.

As used herein, “GM3 synthase deficiency” refers to a neurological disorder that is characterized by recurrent seizures (e.g., epileptic seizures, grand mal seizures, etc.) and cognitive defects (e.g., severe intellectual disability), insomnia, deafness, blindness, and dyskinesia. Typically, subjects having GM3S deficiency comprise one or more mutations in a ST3GAL5 gene. A mutation may be a point mutation, non-sense mutation, non-sense mutation, frameshift mutation, etc. Examples of mutations include mutations at position c.862 (e.g., c.C862T), position c.1063 (e.g., c.G1063A), and positions c.584 and c.601 (e.g., c.G584C and c.G601A).

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to increase the expression of GM3S proteins, to extend the lifespan of a subject, to improve in the subject one or more symptoms of disease (e.g., a symptom of GM3S deficiency), etc. The effective amount will depend on a variety of factors such as, for example, the species, age, weight, health of the subject, and the tissue to be targeted, and may thus vary among subject and tissue as described elsewhere in the disclosure.

As used herein, the term “treating” refers to the application or administration of a composition encoding one or more GM3S proteins to a subject, who has a disease associated with GM3S deficiency, a symptom of a disease associated with GM3S deficiency, or a predisposition toward a disease associated with GM3S deficiency (e.g., one or more mutations in the ST3GAL5 gene), with the purpose to cure, heal, alleviate, relieve, alter, remedy, ameliorate, improve, or affect the disorder, the symptom of the disease, or the predisposition toward a disease associated with GM3S deficiency.

Alleviating a disease associated with GM3S deficiency includes delaying the development or progression of the disease, or reducing disease severity. Alleviating the disease does not necessarily require curative results. As used therein, “delaying” the development of a disease (such as a disease associated with GM3S deficiency) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with GM3S deficiency includes initial onset and/or recurrence.

In some embodiments, administration occurs via systemic injection or direct injection to the central nervous system (CNS). In some embodiments, systemic injection is intravenous injection. In some embodiments, direct injection to the central nervous system (CNS) is intracerebral administration, intrathecal administration, or a combination thereof.

### Administration

The isolated nucleic acids and rAAVs of the disclosure may be delivered to a subject in compositions according to any appropriate methods known in the art. For example, an rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (sec, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, rAAV as described in the disclosure are administered by intravenous injection. In some embodiment, rAAV as described in the disclosure are administered via intracerebroventricular (ICV) injection. Intracerebroventricular injection is an injection technique of substances directly into the cerebrospinal fluid in cerebral ventricles in order to bypass the blood brain barrier. The technique is used in to introduce drugs, therapeutic RNAs, plasmid DNAs, and viral vectors into the CNS. In some embodiments, ICV injection of the rAAV improves survival of the subject. In some embodiments, ICV injection of the rAAV reduces acute liver toxicity associated with rAAV administration. In some embodiments, ICV injection of the rAAV reduces acute liver toxicity as compared to a systemic administration route of the rAAV (e.g., intravenous administration) by at least 10%, at least 20%, at least 30%, at least 40%, at least 50%, at least 60%, at least 70%, at least 80%, at least 85%, at least 90%, at least 95%, at least 99%, or 100% as compared to a subjected receiving the rAAV by a systemic administration route (e.g., intravenous administration). Method of measuring acute liver toxicity are known in the art (e.g., measuring blood ALT/AST level and evaluating clinical symptoms of acute liver toxicity). Any known method for evaluating liver toxicity can be used in the methods described herein.

In some embodiments, the rAAV are administered by intracerebral injection. In some embodiments, the rAAV are administered by intrathecal injection. In some embodiments, the rAAV are administered by intrastriatal injection. In some embodiments, the rAAV are delivered by intracranial injection. In some embodiments, the rAAV are delivered by cisterna magna injection. In some embodiments, the rAAV are delivered by cerebral lateral ventricle injection.

Aspects of the instant disclosure relate to compositions comprising one or more recombinant AAVs, each rAAV comprising a capsid protein and a nucleic acid encoding a transgene, wherein the transgene comprises a nucleic acid sequence encoding one or more GM3S proteins. In some embodiments, each GM3S protein independently comprises or consists of the sequence set forth in any one of SEQ ID NOs: 8-10. In some embodiments, the nucleic acid further comprises AAV ITRs. In some embodiments, the rAAV comprises an rAAV vector represented by the sequence set forth in any one of SEQ ID NO: 1-7, and 11-38, or a portion thereof. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intracerebroventricular (ICV), intramuscular, subcutaneous, intradermal, intratumoral, and other parental routes of administration. In some embodiments, the rAAVs are administered via ICV. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target CNS tissue. In some cases, stable transgenic animals are produced by multiple doses of an rAAV. An effective amount of an rAAV is also dependent on the administration route. In some embodiments, the rAAV is administered via ICV and the effective amount of the rAAV is between about 10⁸ and about 10¹¹ genome copies, between about 10⁹ and about 10¹¹ genome copies, between about 10¹¹ and about 10¹³ genome copies, between about 10¹¹ and about 10¹³ genome copies, between about 1×10¹¹ and about 1×10¹⁵ genome copies, between about 5×10¹¹ and about 5×10¹⁴ genome copies, between about 1×10¹² and about 1×10¹³ genome copies, between about 10¹² and about 5×10¹² genome copies, between about 10¹¹ and about 5×10¹¹ genome copies, between about 5×10¹² and about 10¹³ genome copies, between about 10⁸ and about 10⁹ genome copies (e.g., 1×10⁸ genome copies, 2×10⁸ genome copies, 3×10⁸ genome copies, 4×10⁸ genome copies, 5×10⁸ genome copies, 5×10⁸ genome copies, 6×10⁸ genome copies, 7×10⁸ genome copies, 8×10⁸ genome copies, 9×10⁸ genome copies, 10⁹ genome copies), between about 10⁹ and about 10¹⁰ genome copies (e.g., 1×10⁹ genome copies, 2×10⁹ genome copies, 3×10⁹ genome copies, 4×10⁹ genome copies, 5×10⁹ genome copies, 5×10⁹ genome copies, 6×10⁹ genome copies, 7×10⁹ genome copies, 8×10⁹ genome copies, 9×10⁹ genome copies, 10¹⁰ genome copies), between about 10¹⁰ and about 10¹¹ genome copies (e.g., 1×10¹⁰ genome copies, 2×10¹⁰ genome copies, 3×10¹⁰ genome copies, 4×10¹⁰ genome copies, 5×10¹⁰ genome copies, 5×10¹⁰ genome copies, 6×10¹⁰ genome copies, 7×10¹⁰ genome copies, 8×10¹⁰ genome copies, 9×10¹⁰ genome copies, 10¹¹ genome copies), between about 10¹¹ and about 10¹² genome copies (e.g., 1×10¹¹ genome copies, 2×10¹¹ genome copies, 3×10¹¹ genome copies, 4×10¹¹ genome copies, 5×10¹¹ genome copies, 5×10¹¹ genome copies, 6×10¹¹ genome copies, 7×10¹¹ genome copies, 8×10¹¹ genome copies, 9×10¹¹ genome copies, 10¹² genome copies), between about 10¹² and about 10¹³ genome copies (e.g., 1×10¹² genome copies, 2×10¹² genome copies, 3×10¹² genome copies, 4×10¹² genome copies, 5×10¹² genome copies, 5×10¹² genome copies, 6×10¹² genome copies, 7×10¹² genome copies, 8×10¹² genome copies, 9×10¹² genome copies, 10¹³ genome copies), between about 10¹³ and about 10¹⁴ genome copies (e.g., 1× 10¹³ genome copies, 2×10¹³ genome copies, 3×10¹³ genome copies, 4×10¹³ genome copies, 5×10¹³ genome copies, 5×10¹³ genome copies, 6×10¹³ genome copies, 7×10¹³ genome copies, 8×10¹³ genome copies, 9×10¹³ genome copies, 10¹⁴ genome copies), or between about 10¹⁴ and about 10¹⁵ genome copies (e.g., 1×10¹⁴ genome copies, 2×10¹⁴ genome copies, 3×10¹⁴ genome copies, 4×10¹⁴ genome copies, 5×10¹⁴ genome copies, 5×10¹⁴ genome copies, 6×10¹⁴ genome copies, 7×10¹⁴ genome copies, 8×10¹⁴ genome copies, 9×10¹⁴ genome copies, 10¹⁵ genome copies).

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two-calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and include, for example, addition of surfactants, pH adjustment, salt concentration adjustment, etc. (Sec, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either Intracerebroventricularly, subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use. In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLE

### Example 1

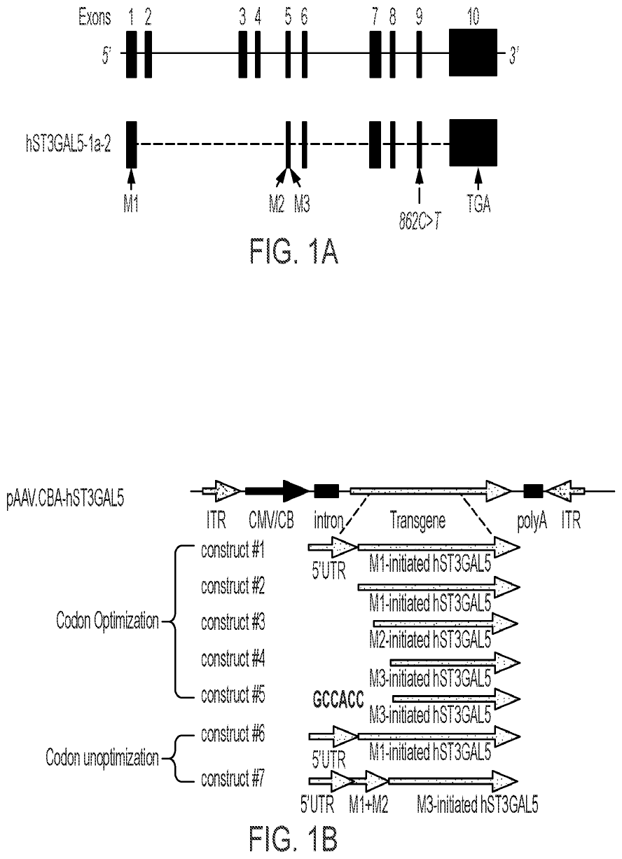

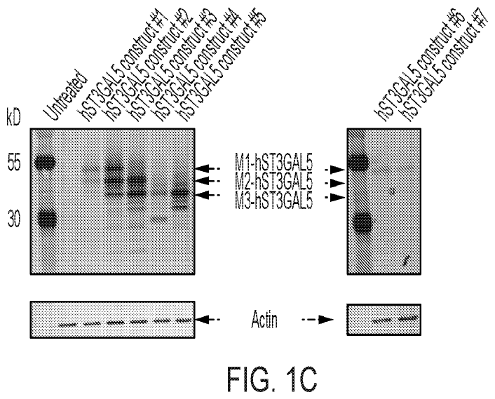

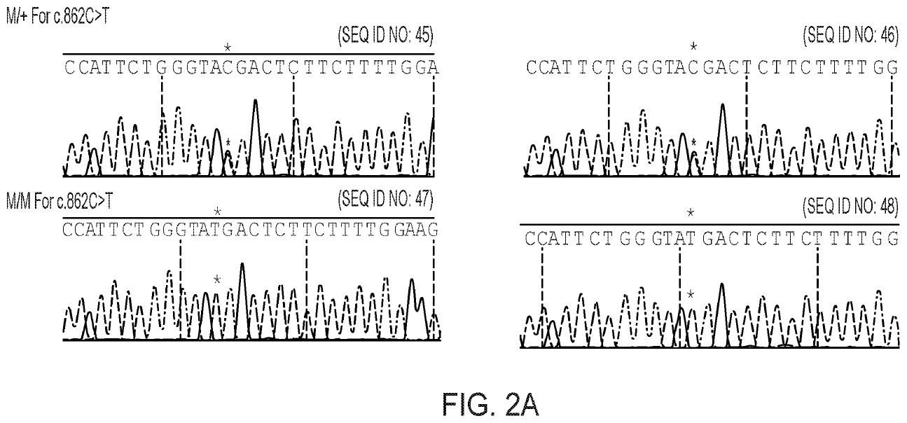

One GM3S mRNA variant (hST3GAL5-1a-2) has been observed to be the most abundant isoform in human tissues, including brain. However, hST3GAL5-1a-2 can produce at least three protein products that initiate from different start codons (FIG. 1A). Therefore, multiple hST3GAL5 constructs that differ in their initiating start codons, codon optimization, and the presence of the full length 5′-UTR (FIG. 1B) were produced.

Expression efficiency of the constructs was investigated by transfecting Hela cells, which normally have no detectable endogenous ST3GAL5 expression. All constructs expressed the desired GM3S isoform. It was noted that constructs having a 5′-UTR exhibited reduced gene expression (FIG. 1C). Constructs without the 5′-UTR were investigated further.

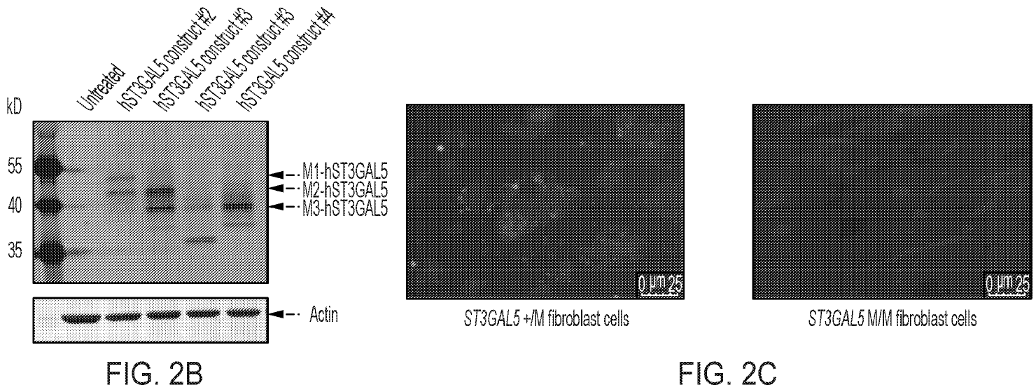

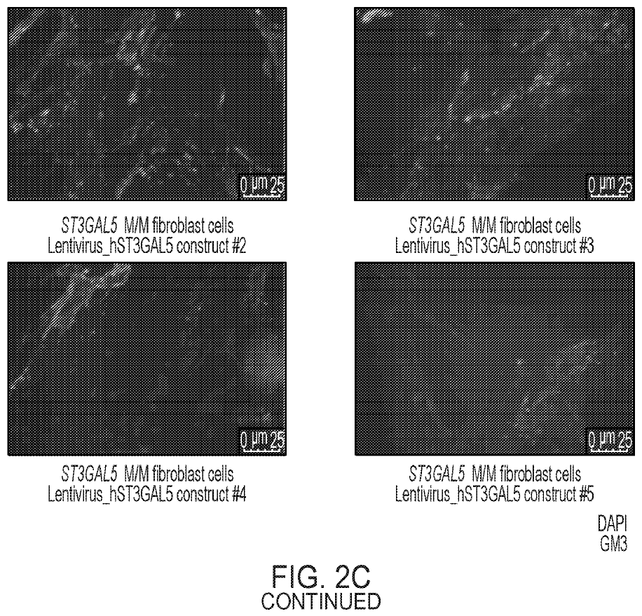

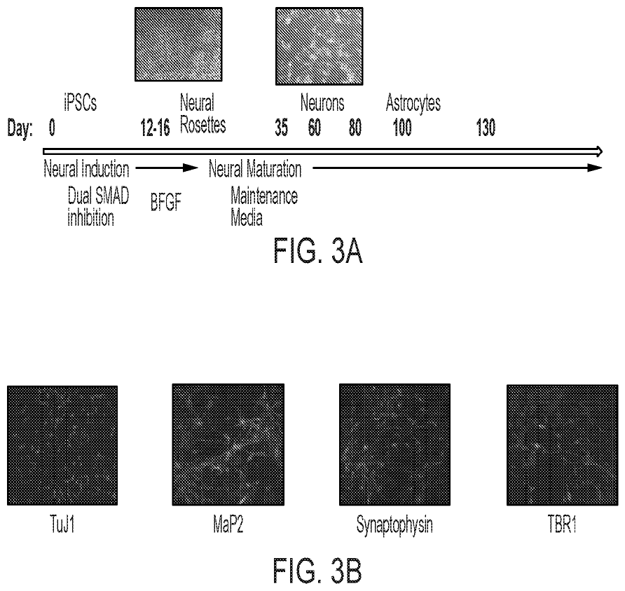

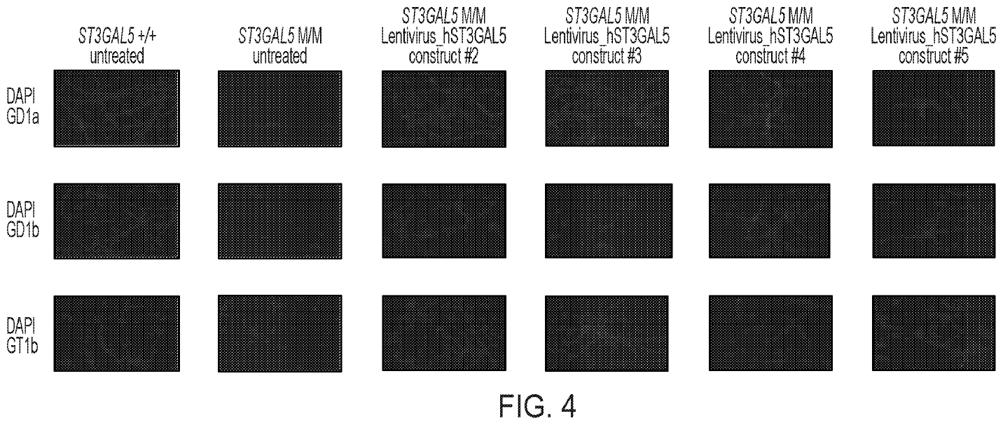

Transfection with ST3GAL5 constructs restored GM3S production in patient-derived fibroblasts (FIGS. 2A-2B). Anti-GM3 antibody assays were performed to assess GM3 expression in patient-derived fibroblasts (FIG. 2C). In addition, human ST3GAL5 cDNAs encoding each isoform of the ST3GAL5 protein were delivered into iPSC-derived cortical neurons from patients with ST3GAL5 deficiency by lentiviral-mediated gene delivery (FIGS. 3A-3B). hST3GAL5 gene replacement in iPSC-derived cortical neurons reconstituted GM3 as well as its major downstream a- and b-series brain gangliosides (e.g., GD1a, GD1b, and GT1b) (FIG. 4). These in vitro data demonstrates the strong therapeutic potential of human ST3GAL5 cDNA gene replacement therapy.

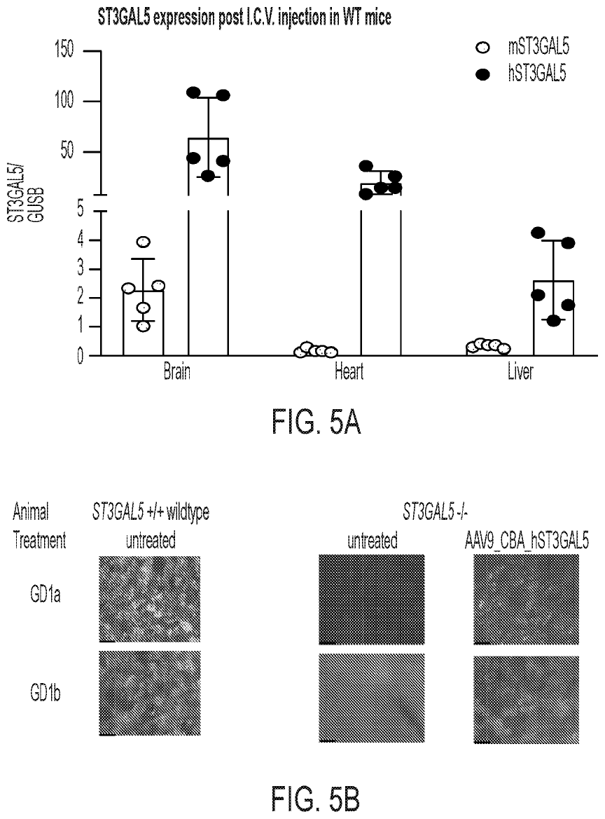

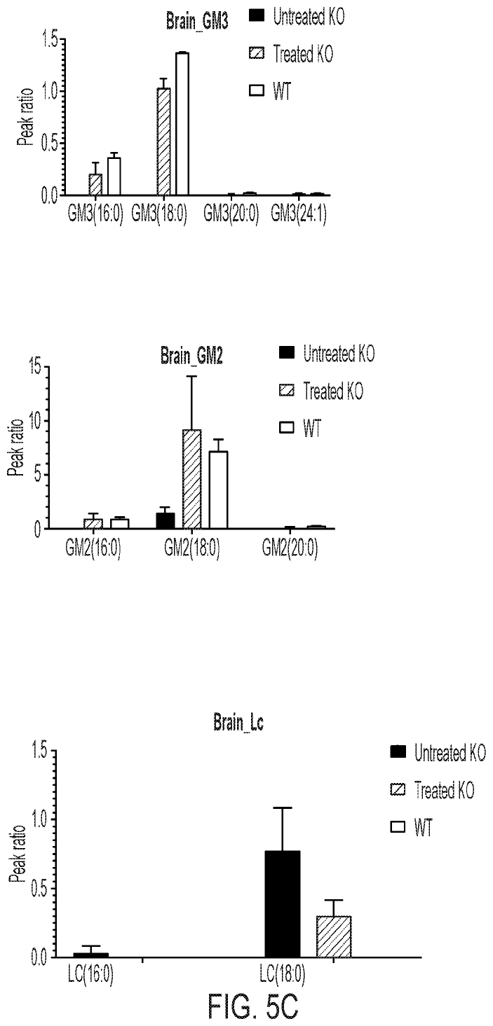

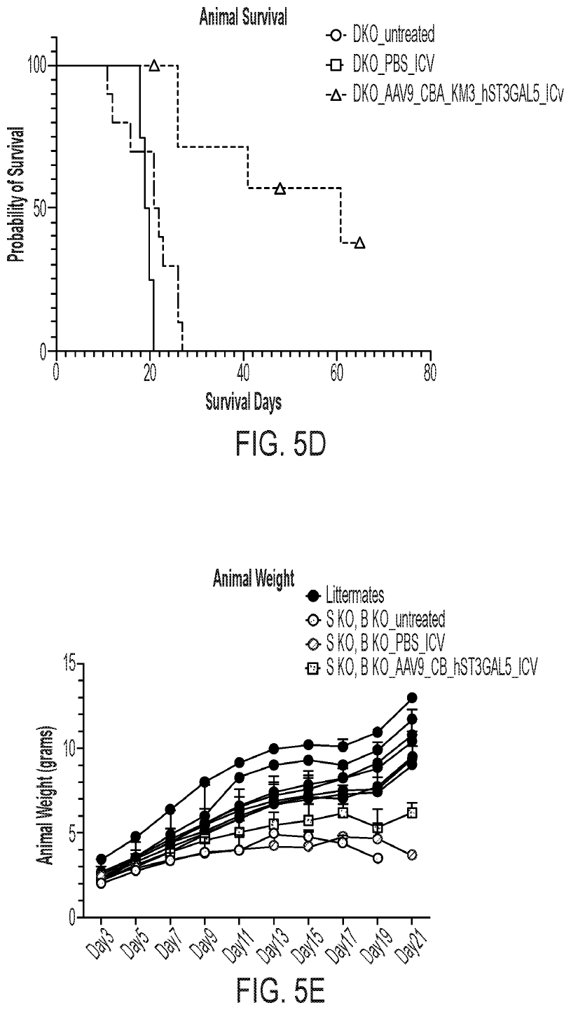

The hST3GAL5 constructs were packaged into AAV9 capsids. To examine the safety and efficacy of transgene expression, the AAVs were injected via intracerebroventricular (ICV) administration to wild type (WT) neonatal C57BL/6 mice. Robust transgene expression in CNS without short-term vector-associated toxicity was observed (FIG. 5A). Furthermore, to assess the therapeutic efficacy of ST3GAL5 gene replacement, AAV9-hST3GAL5 viral particles were delivered to St3gal5^{-/-} B4galnt1 double knock-out (DKO) mice, which are unable to synthesize any cerebral gangliosides and mirror the phenotype of human GM3 synthase deficiency. Direct ICV treatment of neonatal St3gal5 KO mice restored the production of GM3, GM2, GD1a, and GD1b (FIGS. 5B-5C), improved DKO mice physical growth, and extended median survival from 20 days to more than 5 months (FIGS. 5D-5E).

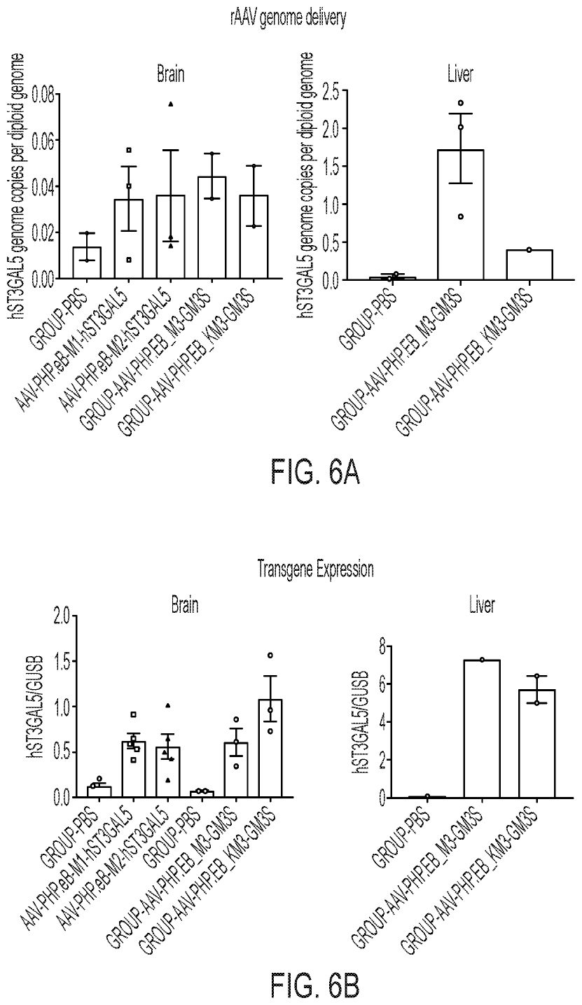

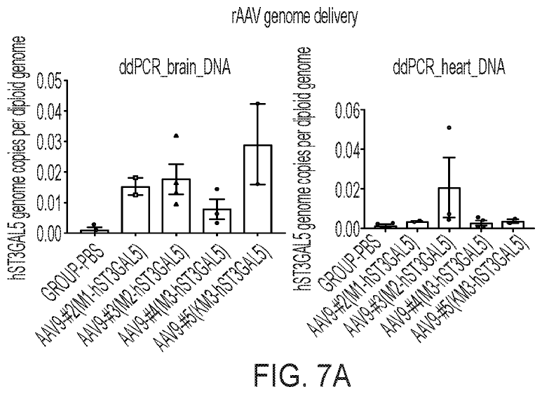

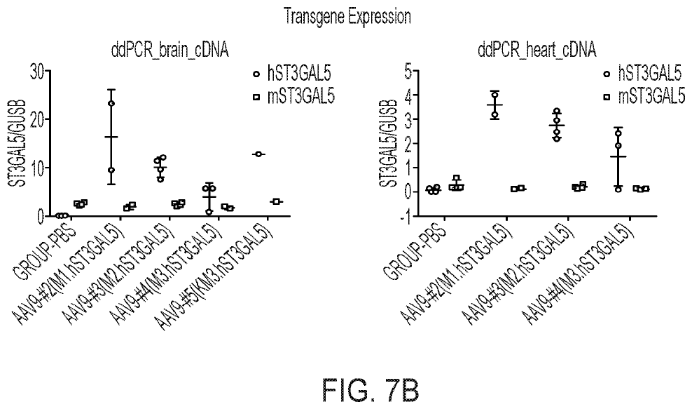

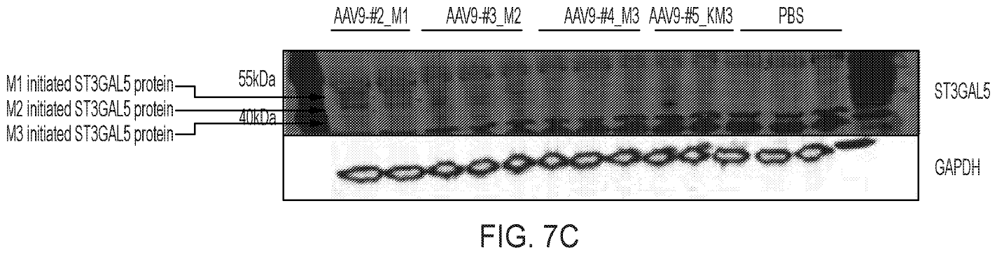

The hST3GAL5 constructs were packaged into AAV9 or AAV-PHP.eB capsids and the AAVs were injected via facial vein into neonatal wild type mice. There was no observed dose-related toxicity. AAV vector delivery and GM3S protein over-expression in the brain were quantified by Droplet Digital PCR (ddPCR) and western blot, respectively (FIGS. 6A-6B), and no short-term vector-associated toxicity (FIG. 7A-7C) was observed.

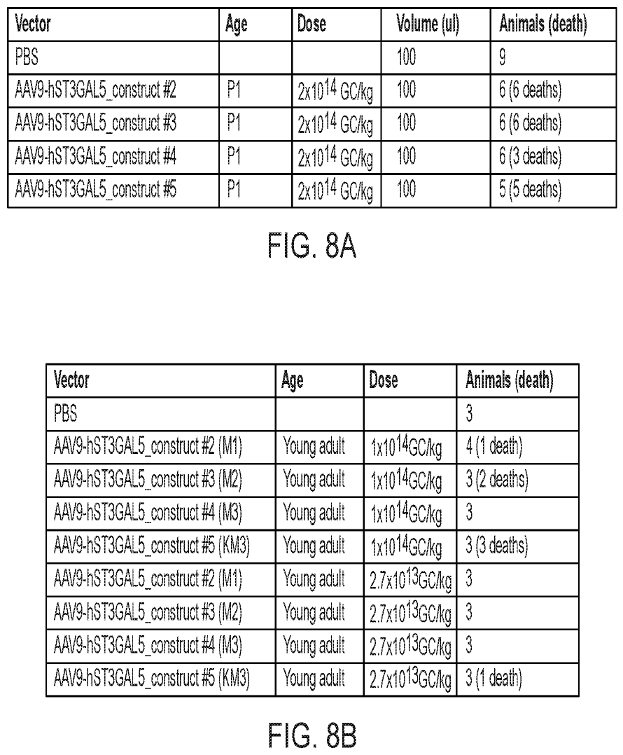

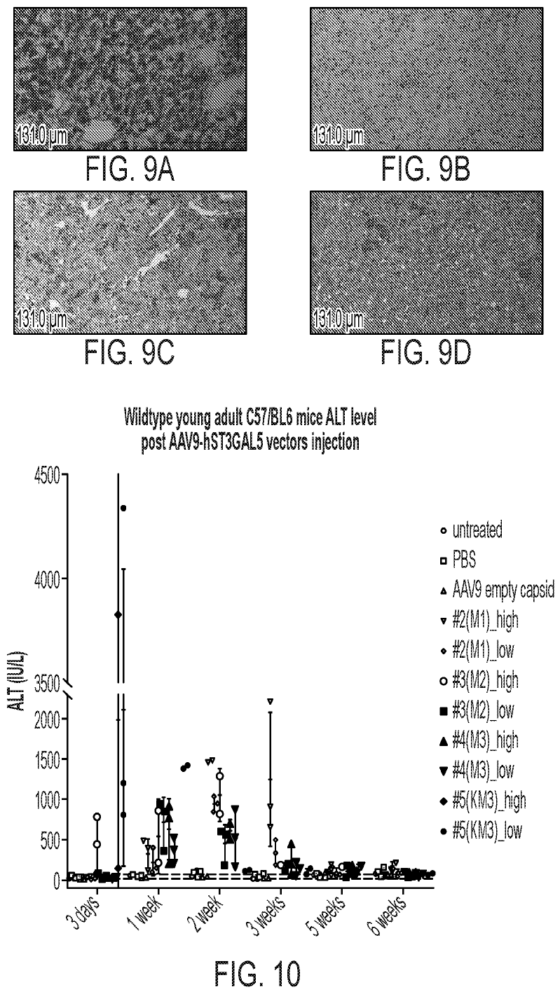

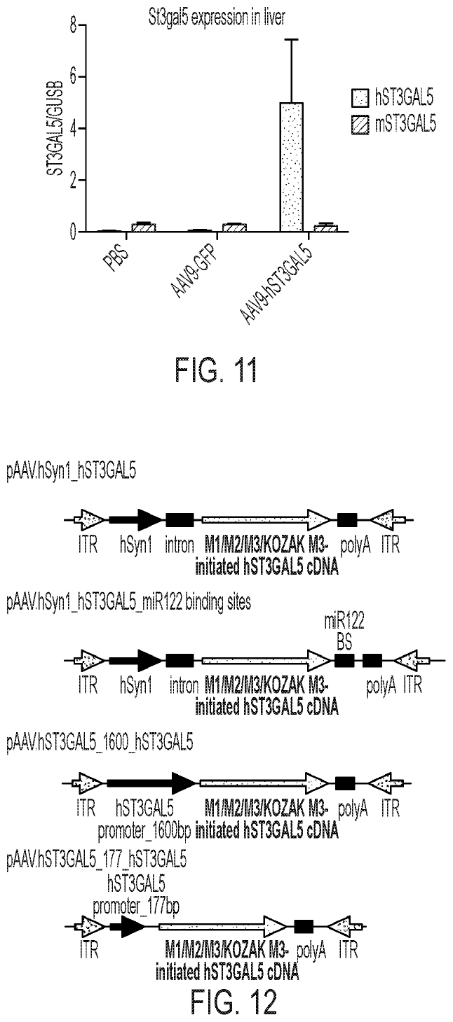

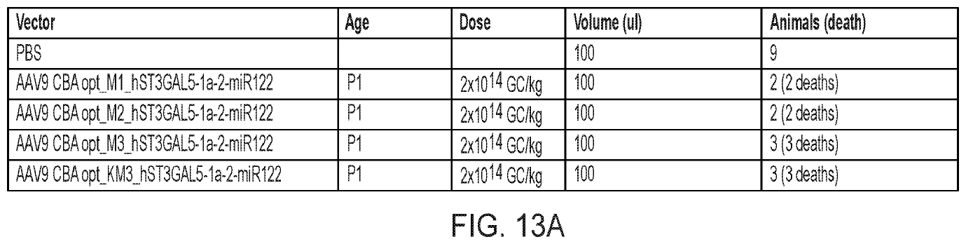

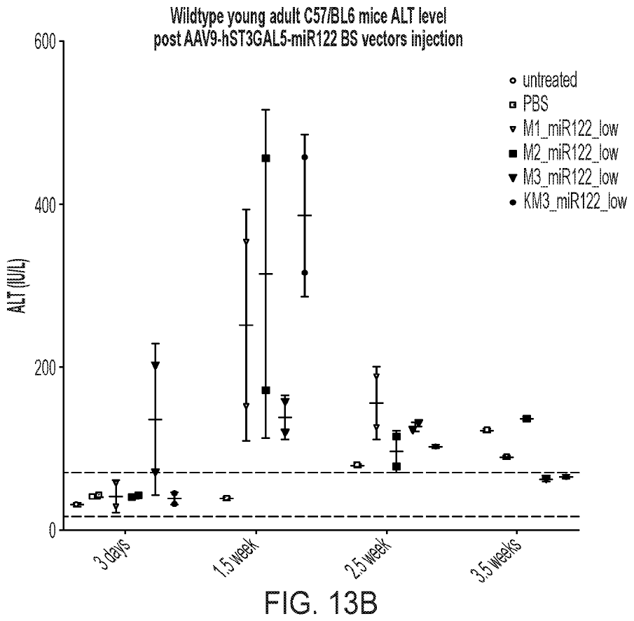

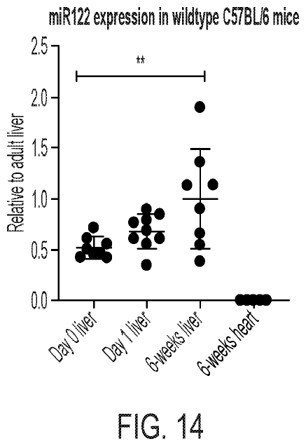

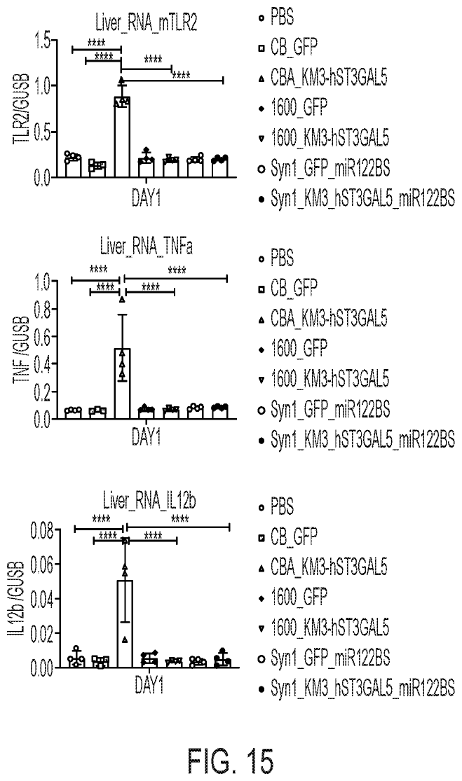

However, surprisingly, administration of AAV9-hST3GAL5 via systemic injection triggered acute liver damage 2-days post injection that culminated in animal death (FIGS. 8A-8B. FIG. 9, and FIG. 10). At the mRNA level, hST3GAL5 expression was 30-fold higher than normal in liver, which may be the cause of the liver damage (FIG. 11). Experiments were designed to de-target hST3GAL5 transgene expression from liver by incorporating endogenous miR-122 (FIG. 12) to the hST3GAL5 vectors. Liver toxicity was reduced in young adult C57/BL5 mice but little improvement was observed in neonatal mice (FIGS. 13A-13B). The animal's survival were largely improved when the rAAVs were co-delivered with miR-122 expression vector. The small RNA analysis suggests the less abundant miR-122 in neonatal liver may contribute to the failure of miR-122 de-targeting (FIG. 14). In addition, a hST3GAL5 vector to include a ubiquitous to neuron-specific promoter—hSyn1 promoter (FIG. 11) in combination with miR-122 binding site—was able to alleviate acute liver toxicity (FIG. 15).

### Example 2

GM3 synthase deficiency (GM3SD) is an infantile-onset lethal epilepsy caused by loss-of-function mutations in the ganglioside GM3 synthase ST3GAL5 gene. Loss of GM3 synthase activity results in the absence of gangliosides, one of the major sphingolipids in the brain, that eventually triggers pervasive neuropathy. In the present study, using ST3GAL5 mutant patient iPSC-derived neurons and St3gal5 knock-out mouse models, ST3GAL5 gene normalization and restoration of the functional products—cerebral gangliosides—was achieved. In addition, hepatic toxicity caused by ubiquitous expression of ST3GAL5 was revealed and a CNS-restricted rAAV gene replacement therapy was established for rescue of the severe neurodevelopmental phenotypes and early lethality in disease mouse models safely, given by both i.e.v. and i.v. routes of administration.

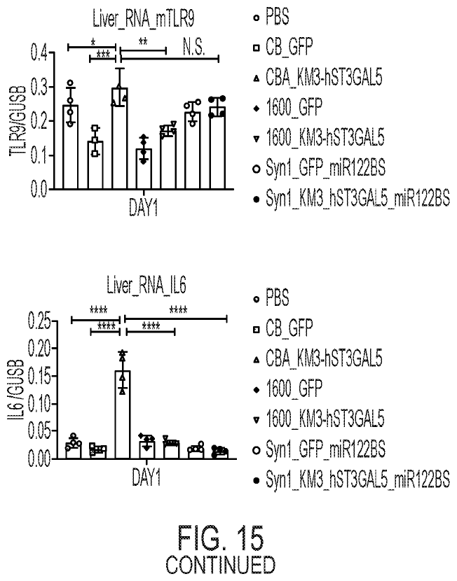

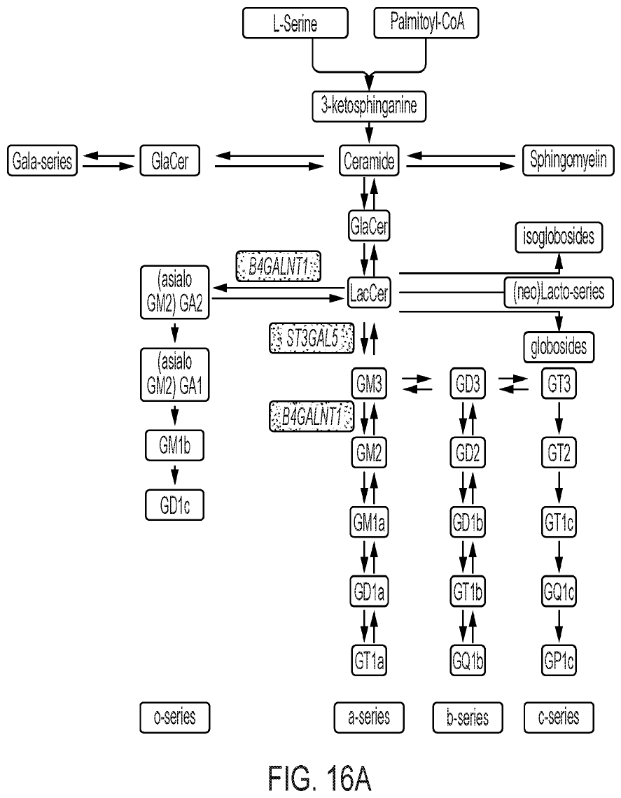

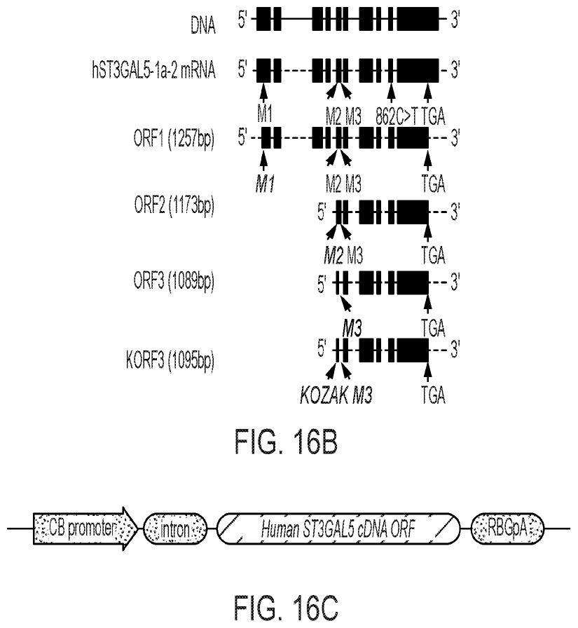

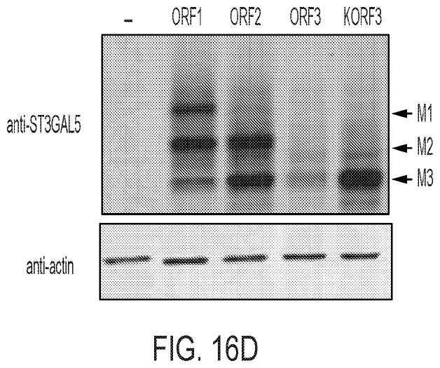

GM3 synthase deficiency (GM3SD) is an autosomal recessive disorder caused by a biallelic premature stop codon mutation, p.R232X, in a ganglioside synthase gene ST3GAL5, which catalyzes the production of GM3 (FIG. 16A). The resulting enzyme deficiency leads to a complete lack of GM3 and its derivatives in all tissues of the body, particularly in the central nervous system (CNS), resulting in reduced survival, motor and cognitive deficits, developmental stagnation, generalized tonic-clonic seizures, and diffused brain atrophy in patients. GM3SD was first found in Old Order Amish communities of North America with an estimated incidence of approximately 1 per 1,200 births. Later on, more cases were also found in non-Amish populations where they present with different mutations in ST3GAL5 but similar biochemical and neurological manifestations. GM3 is a member of the ganglioside family that present in cytosolic membrane and mediate both cis (e.g. architecture of membrane microdomains, the activity of intrinsic membrane protein) and trans (e.g. ligand binding, cell-cell interaction) membrane functions in all mammalian cells. The disrupted ganglioside synthesis affects their interactions with receptors and alters cellular membrane composition, mitochondrial membrane potential, and oxygen consumption, leading to neurotoxicity. Ganglioside replacement therapy with the oral ganglioside supplement G500 powder transiently improves growth and development in patients for the first 1-2 months. But the low absorption and the inability of gangliosides to cross the blood-brain barrier (BBB) may limit the gangliosides supplement therapy to fully rescue the disease symptoms, leading to treatment failure and loss of long-term efficacy.

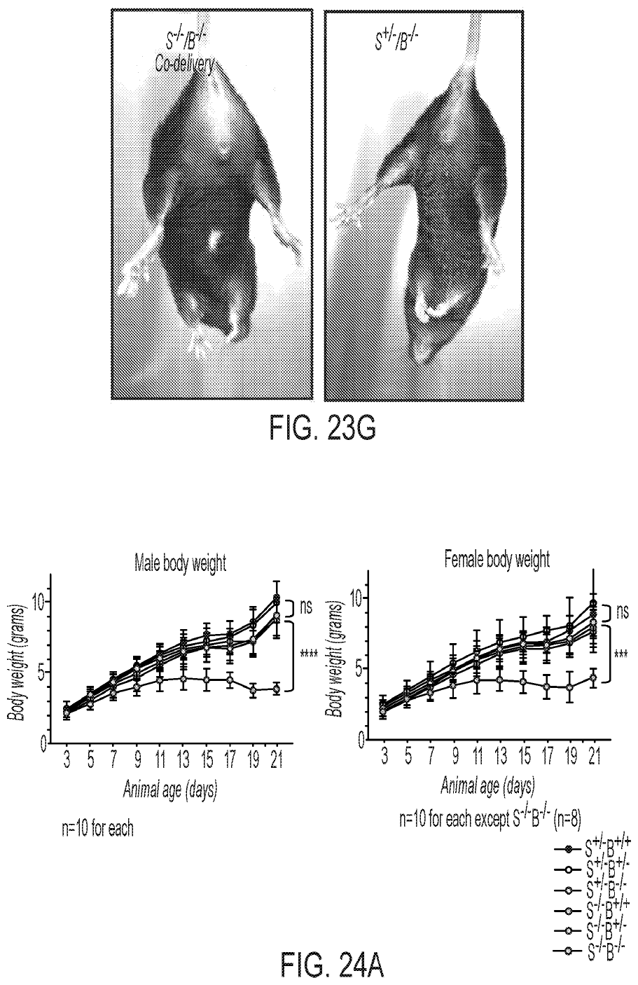

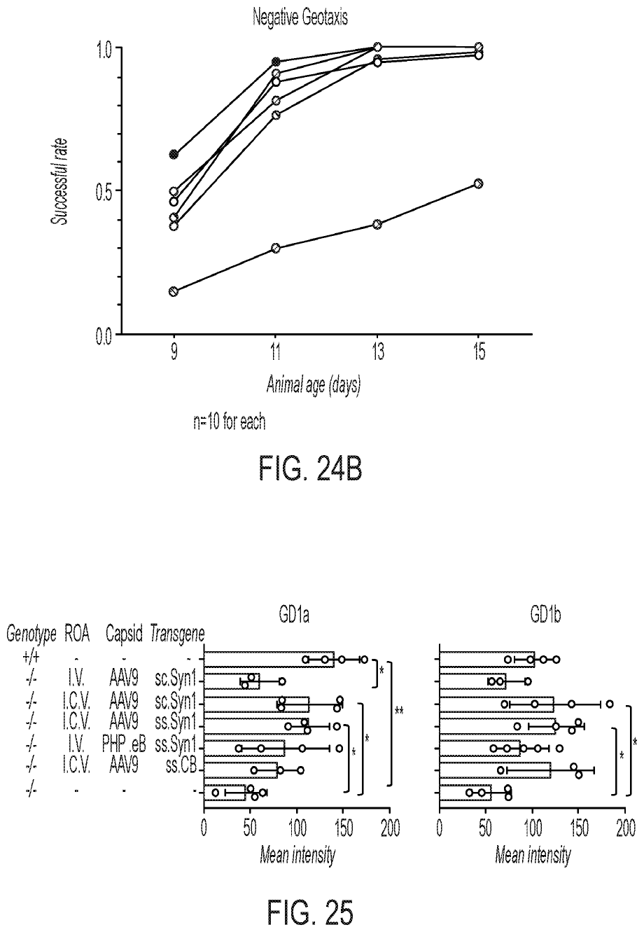

To develop robust therapeutic modalities, proper animal models that can recapitulate GM3SD genetically and phenotypically are required. In contrast to human patients, homozygous St3gal5^{-/-} mice show few clinically relevant phenotypic abnormalities except for insulin hypersensitivity and partial hearing loss regardless of their complete loss of a- and b-series gangliosides that represents the biochemical features in patients. Biochemical defects caused by the loss of GM3 synthase function seem to be compensated by the remaining gangliosides, resulting in minimal physiological alternations in St3gal5^{-/-} mice (FIG. 16A). Indeed, St3gal5^{-/-}/B4galnt1^{-/-} double knock-out mice that are unable to synthesize any ganglioside, recapitulate GM3SD patients' hallmarks such as CNS pathology, developmental delay, motor impairment, and early death (FIG. 24). Thus, St3gal5^{-/-} and St3gal5^{-/-}/B4galnt1^{-/-} mice serve as complementary models for evaluating the therapeutical efficiency both molecularly and phenotypically.

The present disclosure relates to the development of gene replacement therapy for treating GM3SD, which is a monogenic loss-of-function disease. Ideally, the rAAV vector should deliver its therapeutic genome into the target tissue/cells and express its genetic cargo at the appropriate amount and time. AAV capsids with broad tropism to various tissues may be a limiting factor for rAAV-mediated gene therapy for treating GM3SD due to the liver toxicity induced by transgene expression in the liver. Therefore, the present disclosure also contemplates regulated transgene expression with tissue/cell type specificity may be required, through altering the route of administration (ROA) such as local injections, utilizing CNS-favorable viral capsids, and inclusions of cell type-specific promoters and tissue de-targeting microRNA binding sites in the therapeutic genome.

In present disclosure, ST3GAL5 replacement cassettes was constructed and tested for their ability to reconstitute gangliosides in induced pluripotent stem cell (iPSC)-derived cortical neurons from GM3SD patients. Moreover, ST3GAL5 construct was packaged into AAV9 capsid, and the rAAV was delivered to St3gal5^{-/-} and St3gal5^{-/-}/B4galnt1^{-/-} mice via intracerebroventricularly (ICV) delivered, which recapitulated the molecular and phenotypic impairments in human GM3SD respectively. The treatment rescued disease phenotypes including extended lifespan, restored ganglioside production in CNS, improved growth, and partially rescued motor function. However, when delivered systemically, the gene therapy led to hepatic toxicity and acute fatality caused by ectopic expression of ST3GAL5 at supraphysiological levels in the liver. A second-generation of the gene therapy by using a CNS-specific promoter (human Synapsin1) in combination with liver-specific microRNA (miR-122) targeting sequences for both transcriptional and posttranscriptional regulations. The optimized vector eliminated the liver toxicity while preserving the therapeutic effects in the CNS. In addition, we found the currently available St3gal5^{-/-}/B4galnt1^{-/-} mouse model may underestimate the overall therapeutic benefits of the GM3SD gene therapy. Treating the St3gal5^{-/-}/B4galnt1-1-mice with co-injection of ST3GAL5 and B4GALNT1 accomplished complete normalization of the disease animals. Furthermore, therapeutic outcomes through systemic delivery of the second-generation vector using PHP.cB capsid was observed.

### ST3GAL5 Transgene Design and In Vitro Expression

ST3GAL5-1a-2 (NM_003896) is the most abundant messenger RNA (mRNA) among four ST3GAL5 mRNA variants in the human brain (FIG. 16B), therefore this variant was selected for further vector development. The first AUG start codon in ST3GAL5-1a-2 is in a weak translation initiation context (AUUAGUAUGC)(SEQ ID NO: 44), most ribosomes therefore skip the first AUG and recognize either one of the two downstream AUG as the start codon. As a result, there are three ST3GAL5 protein isoforms differing in their N-termini (FIG. 16B). Replacement constructs carrying each open reading frame corresponding to each of the three isoforms of ST3GAL5 were designed (FIG. 16B). The codon-optimized transgenes were cloned into a ubiquitous expression cassette driven by chicken beta-actin (CB) promoter with an intron (FIG. 16C), and transfected into HeLa cells to confirm protein expression. The expression of the shortest construct (M3) was weak, and adding Kozak sequence GCCACC (construct KORF3) greatly enhanced expression (FIG. 16D).

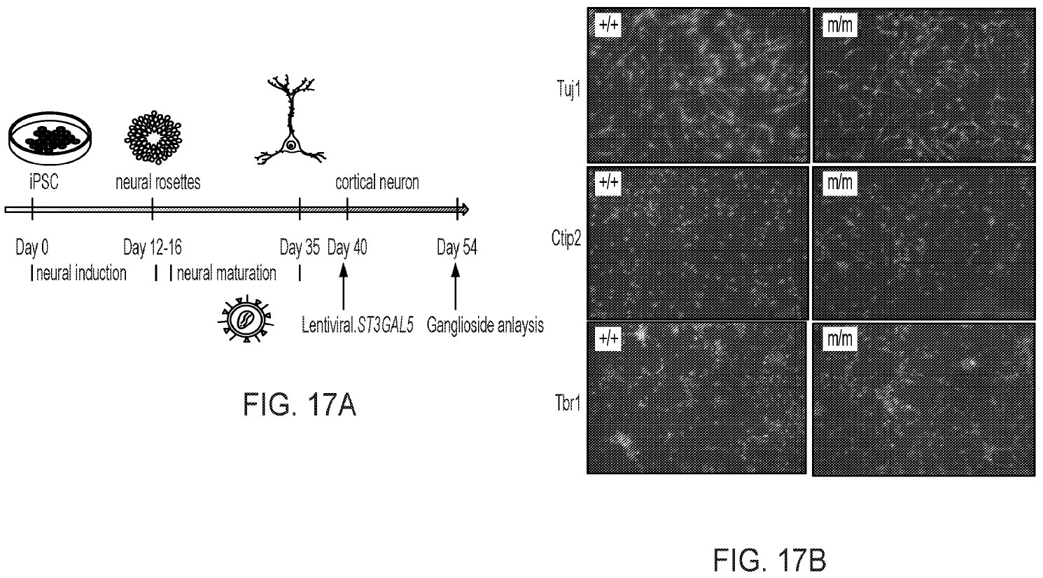

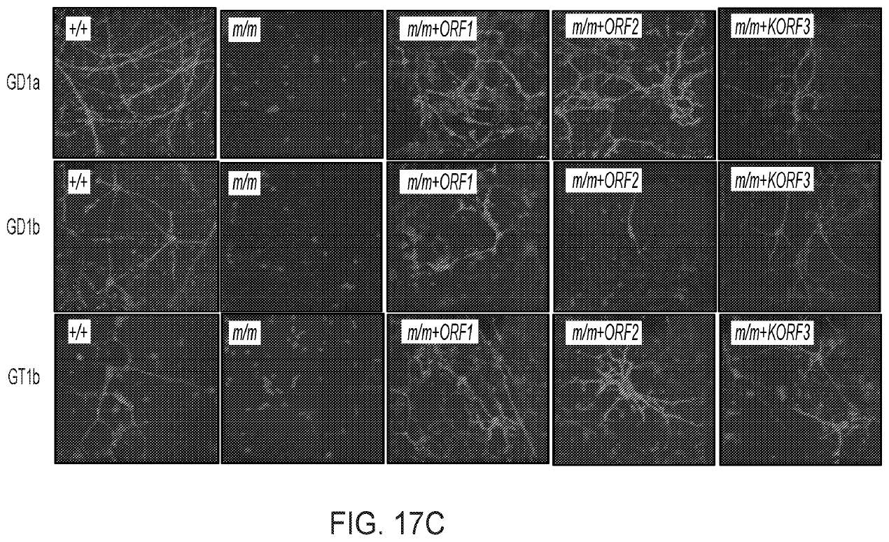

Next, whether these ST3GAL5 constructs could function in ganglioside synthesis in cultured neurons was evaluated. To this end, normal (ST3GAL5+/+) and patient (ST3GAL5E332K/E332K) iPSCs were differentiated into cortical neurons (FIGS. 17A-17B), and infected with lentiviral vectors expressing different ST3GAL5 isoforms for 14 days. While the major brain gangliosides GD1a, GD1b, and GT1b were absent in untreated patient neurons, they were restored following transduction of any ST3GAL5 isoform (FIG. 17C). The KORF3 transgene design was selected for in future development, because its small gene size (1,095 base pairs) is amenable to the self-complementary AAV vector design that can further enhance vector potency but has limited packaging capacity (<2.5 kb).

Intracerebroventricular (ICV) Injection of rAAV9-ST3GAL5.v1 Improved Biochemical and Phenotypic Abnormalities in GM3SD Mouse Models

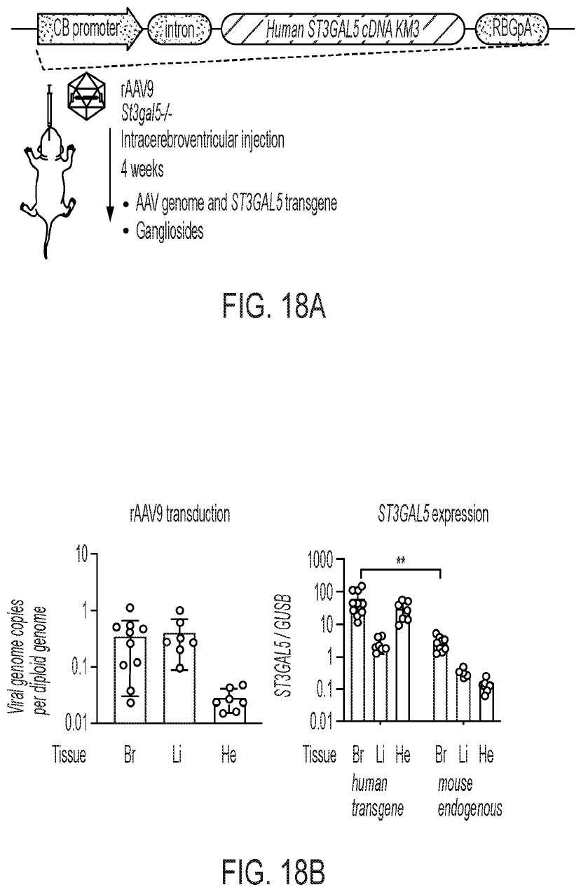

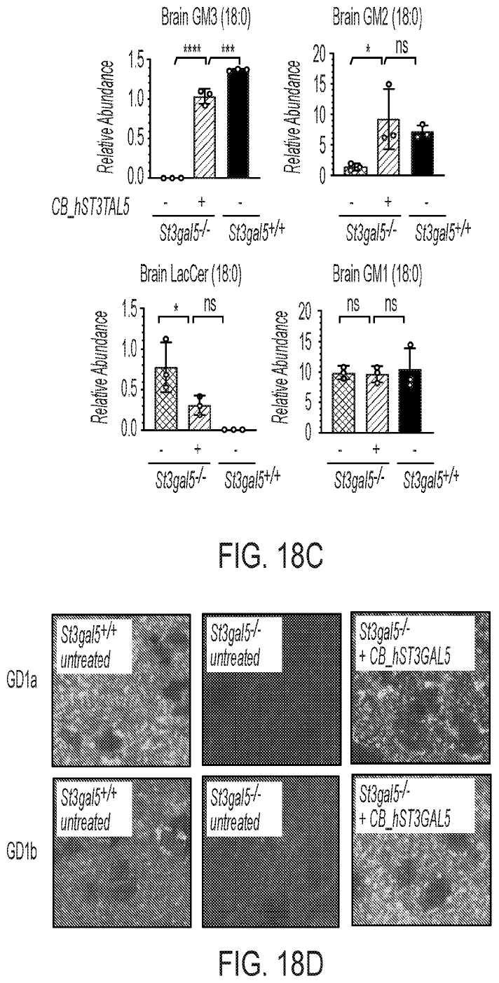

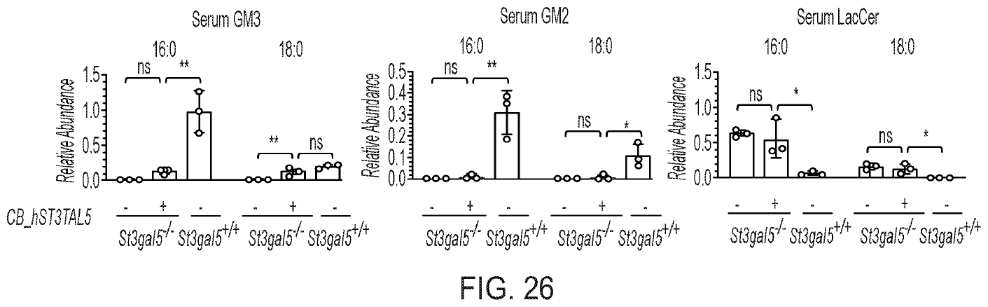

rAAVs comprising AAV9 capsid and the CB promoter-driven ST3GAL5 construct (rAAV9-ST3GAL5.v1) were used to assess therapeutic efficacy in mice following in vivo delivery. St3gal5^{-/-} mice was treated by unilateral ICV injection of 3×10¹⁰ genome copies (gc)/pup at postnatal day 1 (P1), and euthanized at 4 weeks post-injection (FIG. 18A). The ST3GAL5 transgene was detected in the brain, liver, and heart, resulting in mRNA expression exceeding the endogenous mouse St3gal5 mRNA levels (FIG. 18B). The gangliosides undetectable in the St3gal5^{-/-} brain was restored to or close to WT levels, including GM3 and its derivatives GM2, GD1a, and GD1b, concomitant with substantial clearance of the precursor Lactosylceramide (LacCer) (FIGS. 18C-18D, and FIG. 25). However, circulating ganglioside profile was barely restored, likely because gene delivery to peripheral tissues was insufficient by ICV injection (FIG. 26). Since the human brain contains 10- to 30-fold more gangliosides than any other tissue in the body, it is possible that ganglioside restoration in the CNS should suffice to eliminate the neurological deficits, the major disease manifestation in patients.

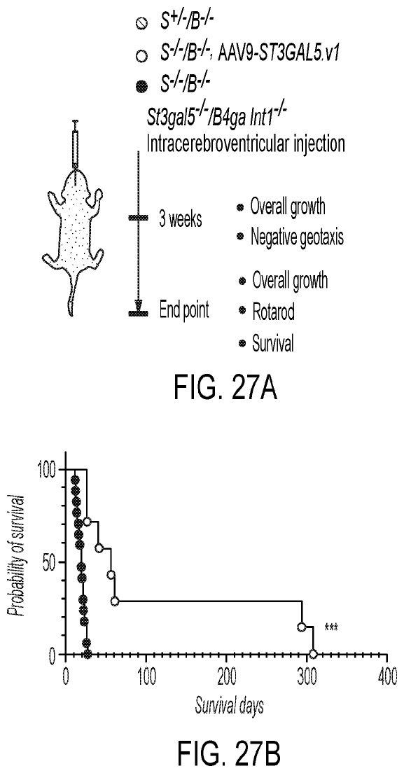

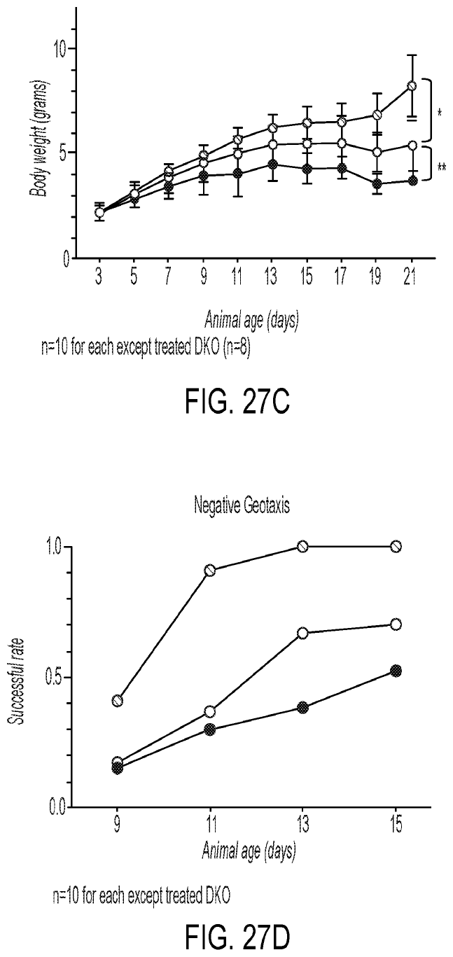

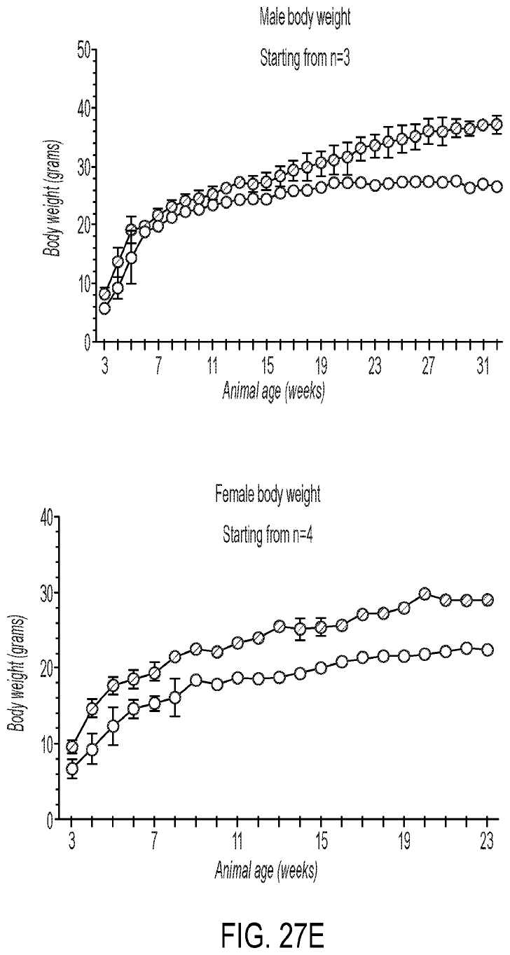

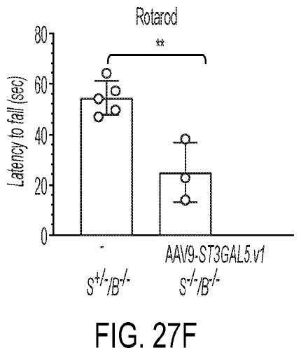

Because the St3gal5^{-/-} mice do not exhibit neurological deficits despite biochemical defects, an St3gal5^{-/-}/B4galnt1^{-/-} mouse model that recapitulates key GM3D disease phenotype, including premature lethality, motor function defects, and neuropathology, and benchmarked to the St3 gal^{+/-}/B4galnt1^{-/-} mice that do not show such phenotype (FIG. 27A). The same P1 ICV treatment regimen to St3gal5^{-/-}/B4galnt1^{-/-} mice significantly extended survival up to 300 days (median survival: untreated, 18-days; treated, 56-days) (FIG. 27B), improved growth, and partially restored motor function as assessed by the negative geotaxis test, although the gene therapy treatment did not fully rescue the St3gal5^{-/-}/B4galnt1^{-/-} mice (FIGS. 27C-27F). Taken together, these results demonstrated that neonatal ICV injection of rAAV9-ST3GAL5.v1 was well tolerated in mice, restored ganglioside production in the St3gal5^{-/-} mice, and partially alleviated the GM3D disease phenotype in the St3gal5^{-/-}/B4galnt1^{-/-} mice.

Systemic Delivery of rAAV9-ST3GAL5.v1 Caused Liver Toxicity

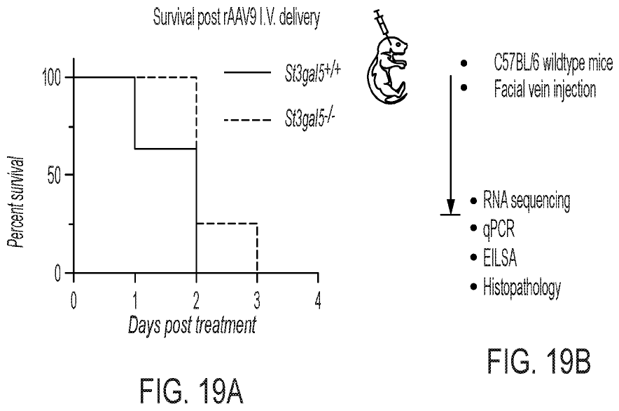

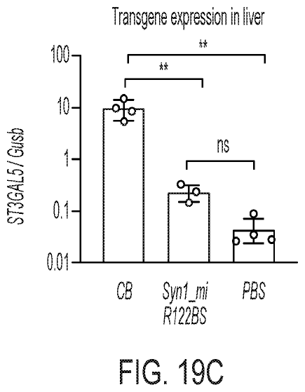

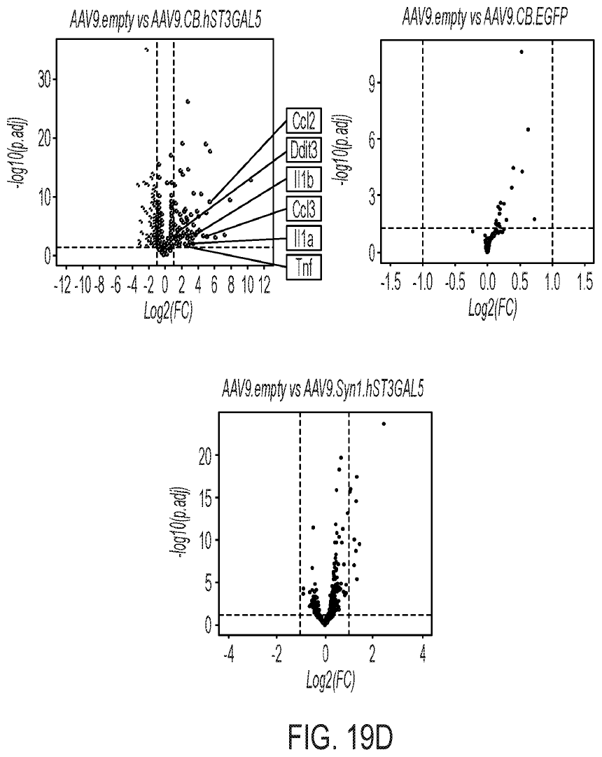

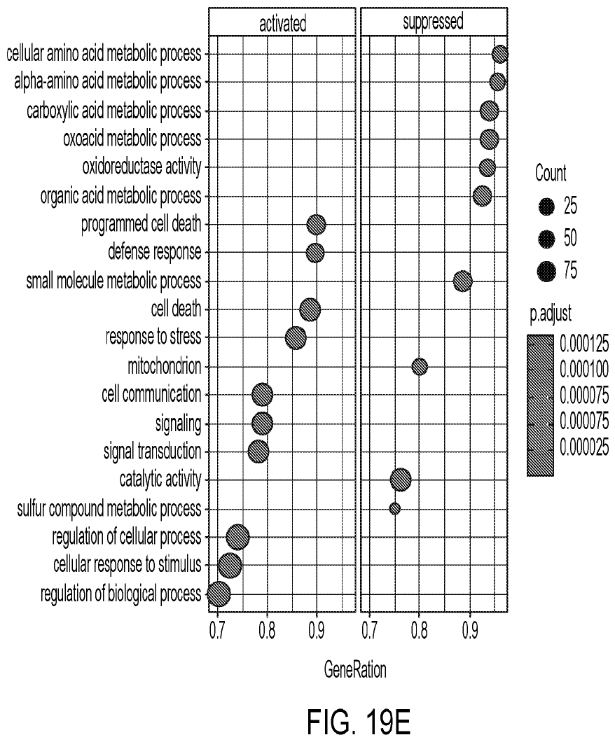

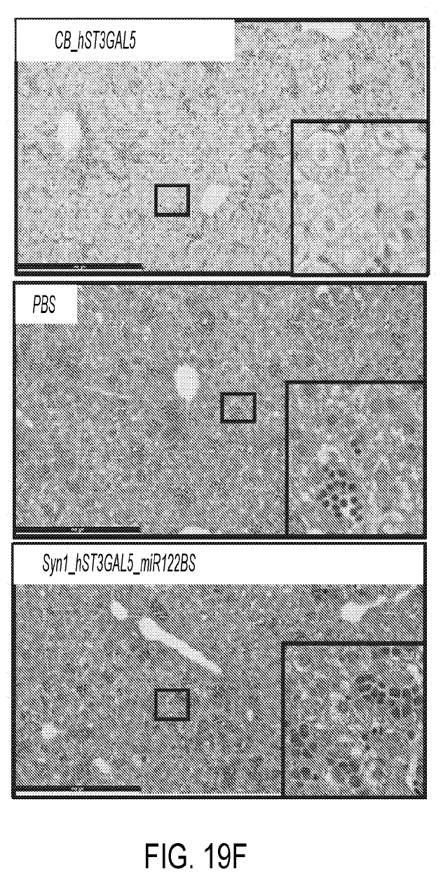

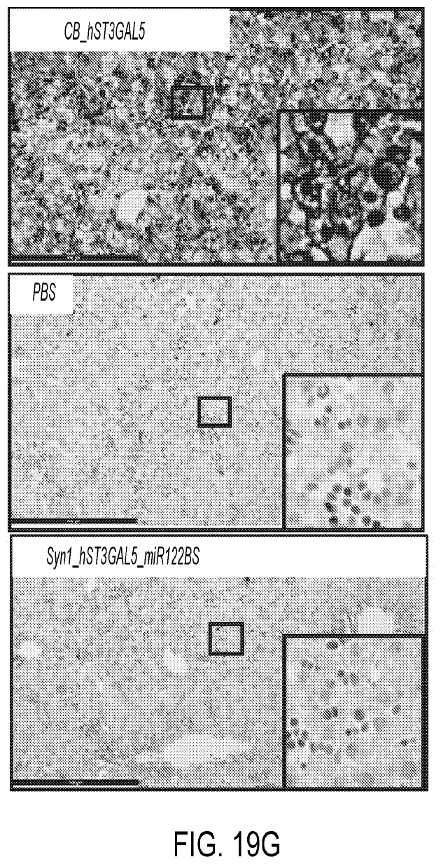

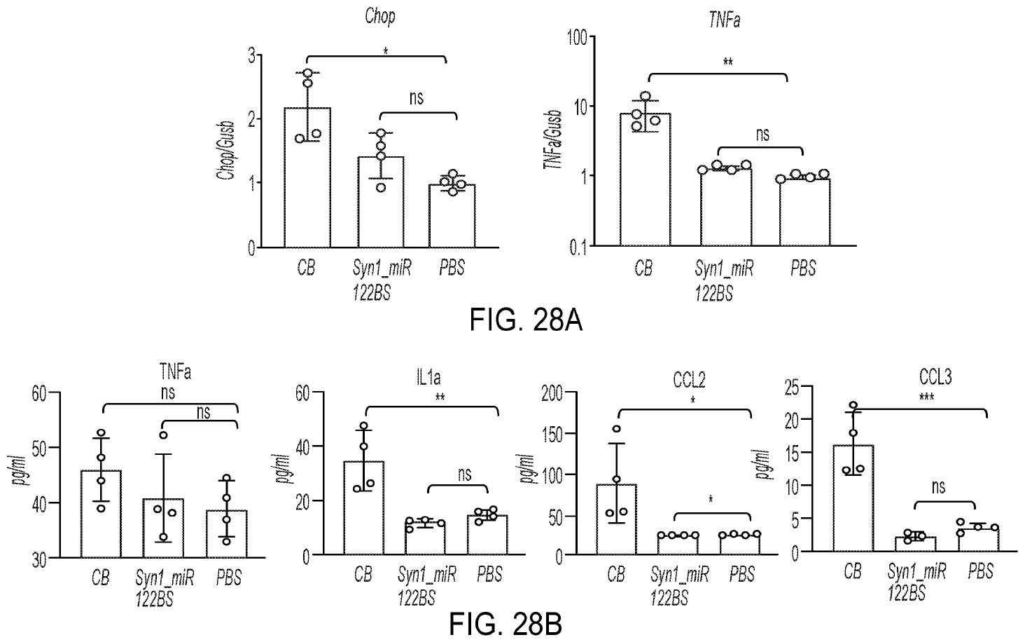

While ICV injection considerably rescued disease phenotype in mice, systemic delivery that broadly distributes AAV9 vector throughout the brain may be advantageous for this whole-brain disease. Therefore, P1 St3gal5^{-/-} pups were treated by facial vein injection of 3×10¹¹ gc/pup; St3gal5^{+/+} littermates were also treated, serving as controls. However, acute lethality was observed within 3 days post injection regardless of genotype (FIG. 19A). In order to understand fatality, wildtype pups were injected with rAAV9-ST3GAL5.v1, rAAV9.EGFP, AAV9.empty or PBS (FIG. 19B). Indeed, it was found that ST3GAL5 expression in the liver was over 100-fold higher than the endogenous mouse St3gal5 (FIG. 19C), which led to the activation of cellular death and defense response (FIGS. 19D-19E). The RNA seq results with qPCR and ELISA found consistent pro-inflammatory cytokines activation (TNFα, IL1α, CCL2, and CCL3) (FIGS. 28A-28B). More importantly, the molecular changes were in line with histopathological phenotypes in the liver including hepatocyte swelling (FIG. 19F) and cell death (FIG. 19G). Therefore, it is possible that the overexpression of hST3GAL5 transgene triggered liver inflammation and toxicity and in turn caused animal death.

Optimized Construct with Spatial Regulation Eliminated Liver Toxicity Associated with Systemic Administration

It was hypothesized that CNS-restricted and liver de-targeted expression of the transgene could retain therapeutic efficacy and meanwhile eliminate liver toxicity. Therefore, a spatially regulated expression cassette that included human Synapsin 1 (Syn1) promoter for neuronal expression regulation at transcriptional level was designed, and miR-122 binding sites were added in the 3′ untranslated region (UTR) for post-transcriptional liver-specific expression silencing (FIG. 19C). This construct was named as ST3GAL5.v2, and packaged into AAV9 capsid. Following the same P1 facial vein treatment in WT pups, transgene expression from rAAV9-ST3GAL5.v2 was greatly reduced in the liver, and all animals survived without inflammation, histopathology, and transcriptomic changes in the liver (FIGS. 19A-19G, and FIG. 28).

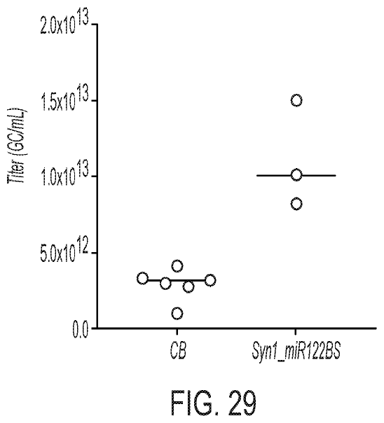

Interestingly, packaging rAAV9-ST3GAL5.v1 always resulted in low titers (1 to 4×10¹² gc/mL), likely due to transgene expression and toxicity in HEK293 cells during vector production. In contrast, rAAV9-ST3GAL5.v2 was routinely produced at higher titers of 0.8 to 1.5×10¹³ gc/mL (FIG. 29). Therefore, by tuning tissue specificity, the v2 construct design eliminated both hepatotoxicity and the manufacturing bottleneck.

ICV Injection of rAAV9-ST3GAL5.v2 Improved Biochemical and Phenotypic Abnormalities in GM3SD Mouse Models

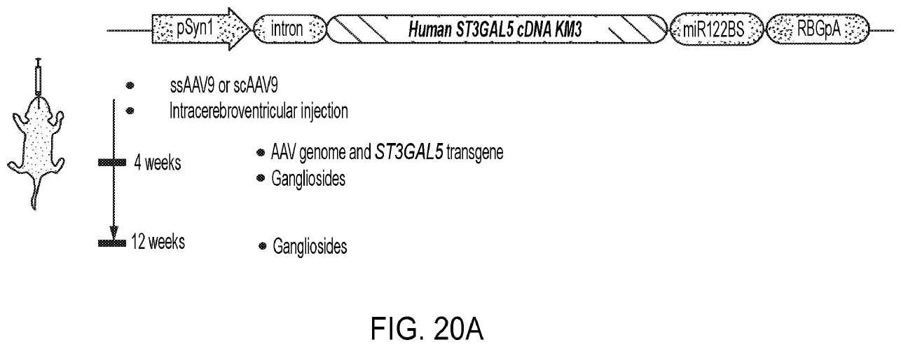

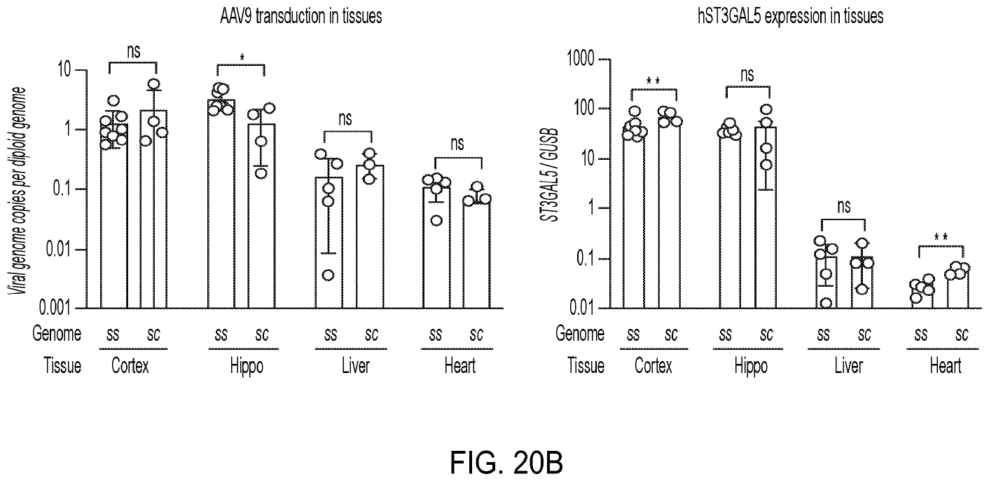

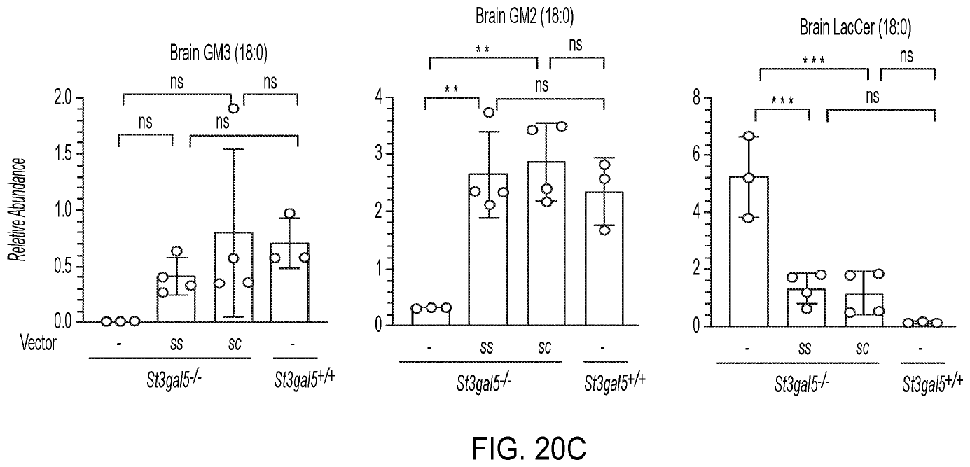

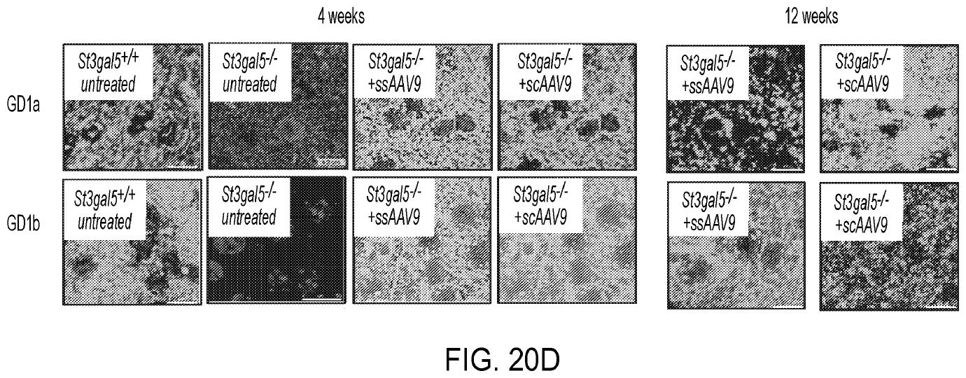

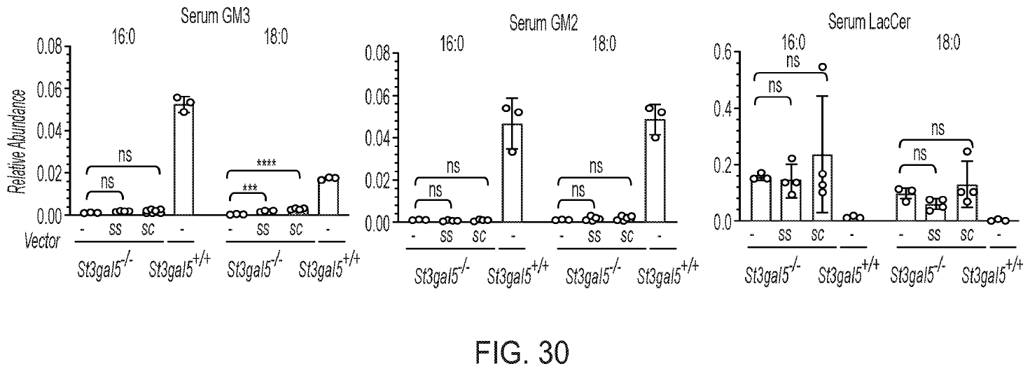

To enhance expression, the ST3GAL5.v2 construct was cloned in self-complementary (sc) configuration that can lead to faster and stronger transgene expression than a single-stranded (ss) form (FIG. 20A), and generated scAAV9 vector. Following P1 ICV injection in St3gal5^{-/-} mice, scAAV9- and ssAAV9-ST3GAL5.v2 led to comparable transgene expression in the hippocampus at 4 weeks post injection, whereas scAAV9 outperformed ssAAV9 in the cortex (FIG. 20B). Both vectors normalized brain ganglioside profile in the St3gal5^{-/-} mice up to 12 weeks post injection (FIGS. 20C-20D), and neither vector corrected circulating gangliosides, consistent with the neuron-specific transgene expression design (FIG. 30).

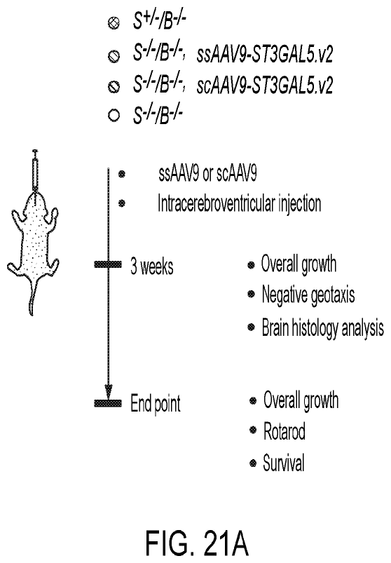

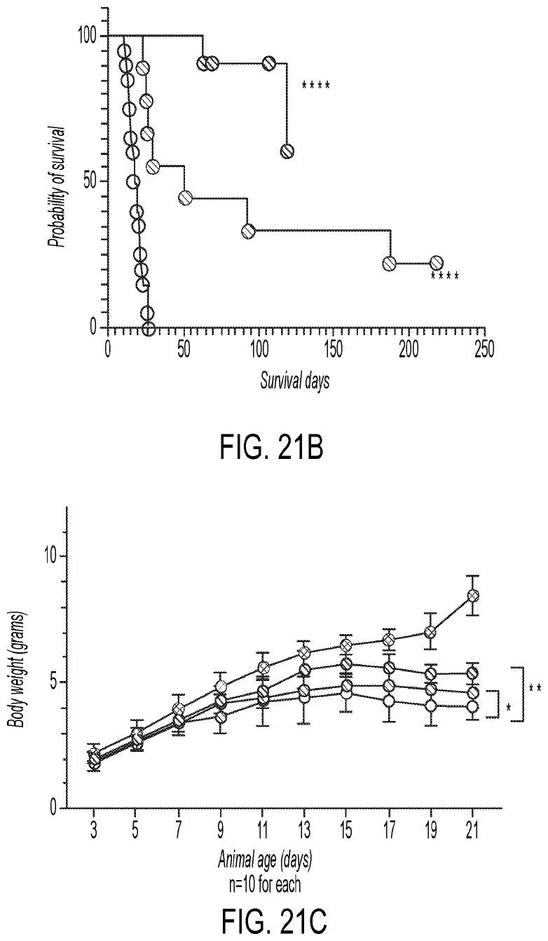

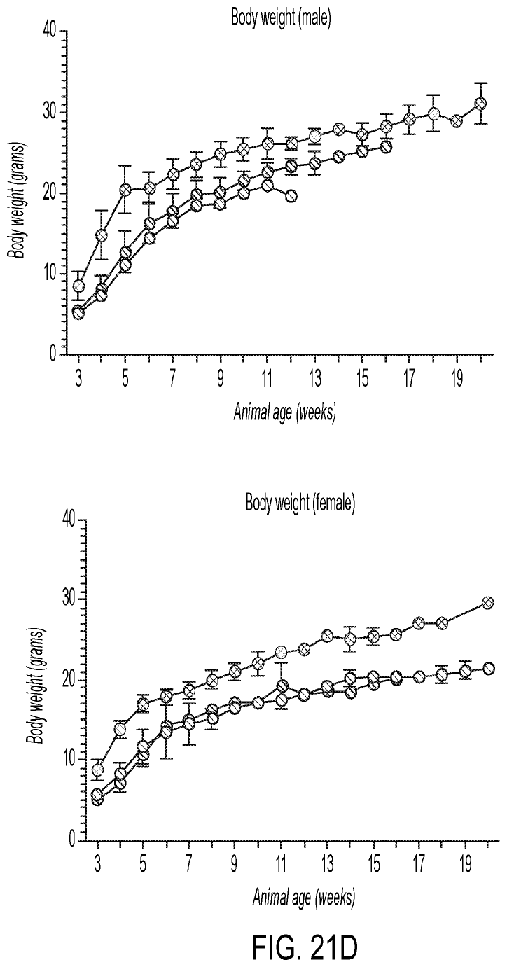

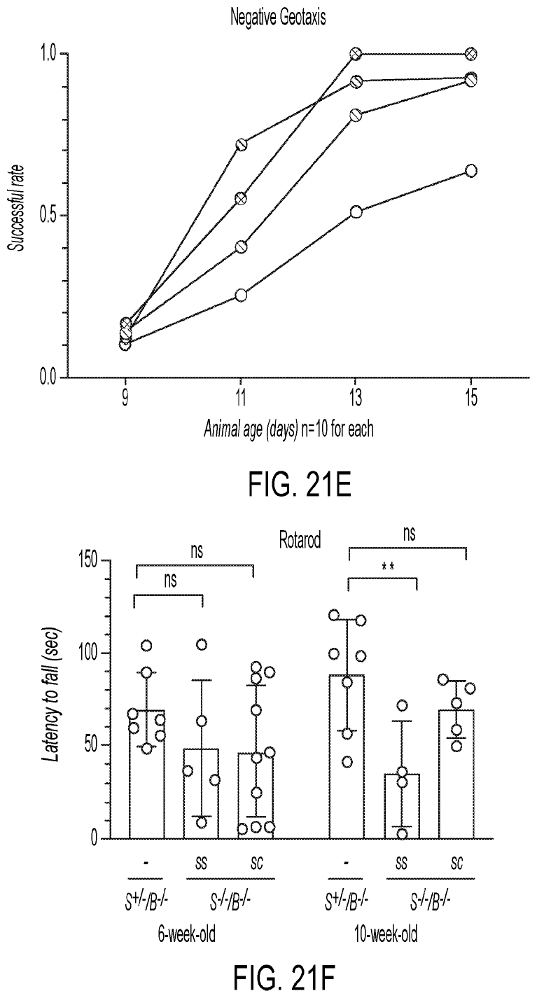

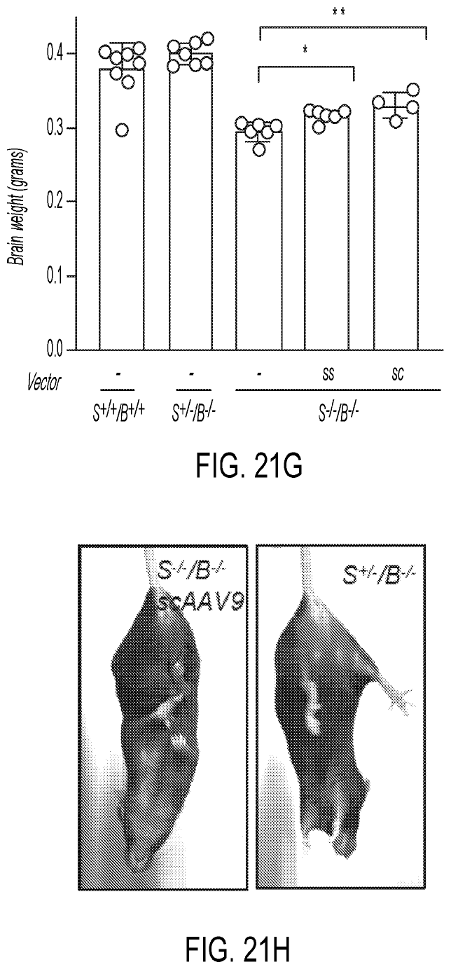

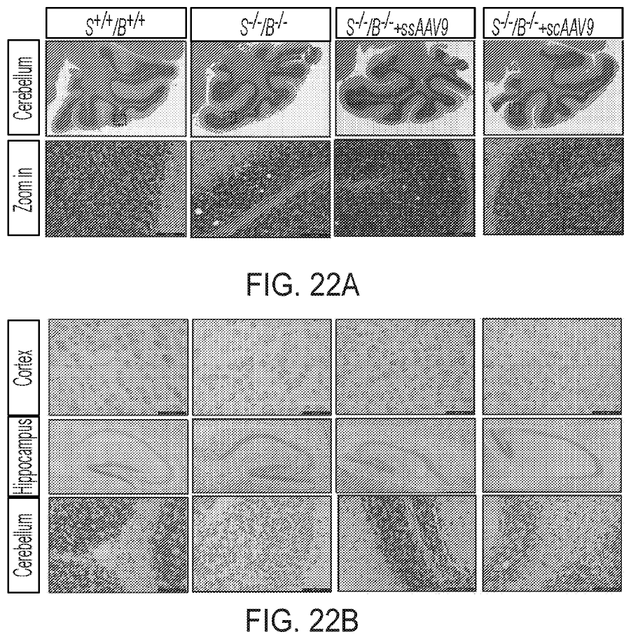

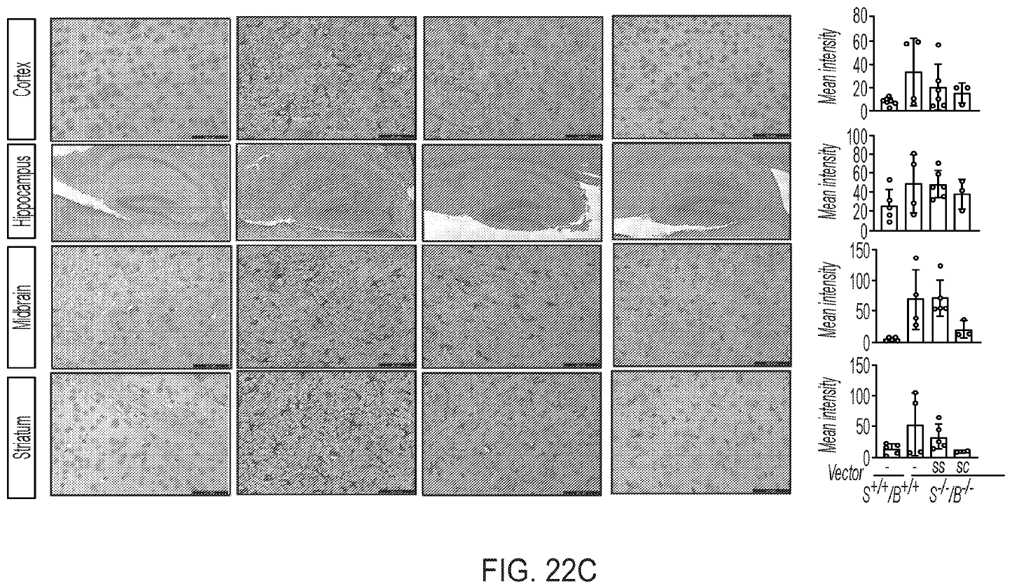

Next, St3gal5^{-/-}/B4galnt1^{-/-} pups were treated by the same P1 ICV injection of scAAV9- or ssAAV9-ST3GAL5.v2 to assess phenotypic rescue (FIG. 21A). The treatment significantly extended animal survival (median survival: untreated, 19-days; ssAAV9, 51-days; scAAV9: 100.5-days) (FIG. 21B), partially restored brain and body weight (FIGS. 21C-21D), and largely normalized motor function as revealed by negative geotaxis test and rotarod test (FIGS. 21E-21F). Importantly, the treatment greatly improved weight (FIG. 21G) and several histopathological changes in the brain, including vacuolation in the cerebellum (FIG. 22A), massive cell death (FIG. 22B), and astrogliosis (FIG. 22C) across multiple regions; scAAV9 consistently outperformed ssAAV9 in all histopathology assessment, likely due to its more rapid and higher transgene expression.

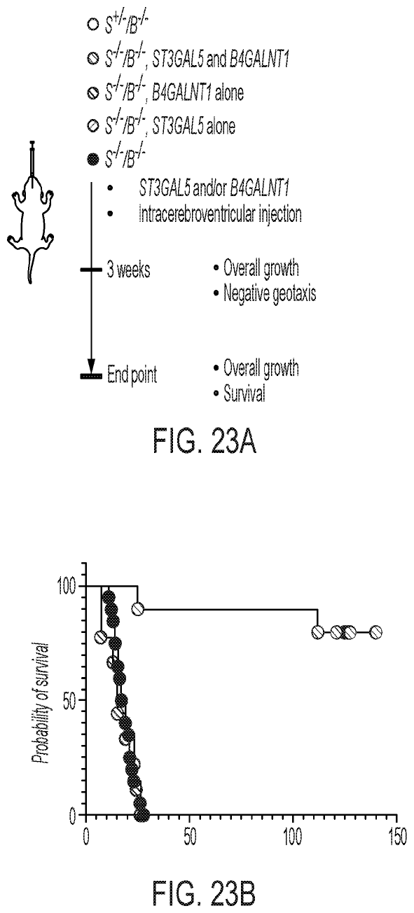

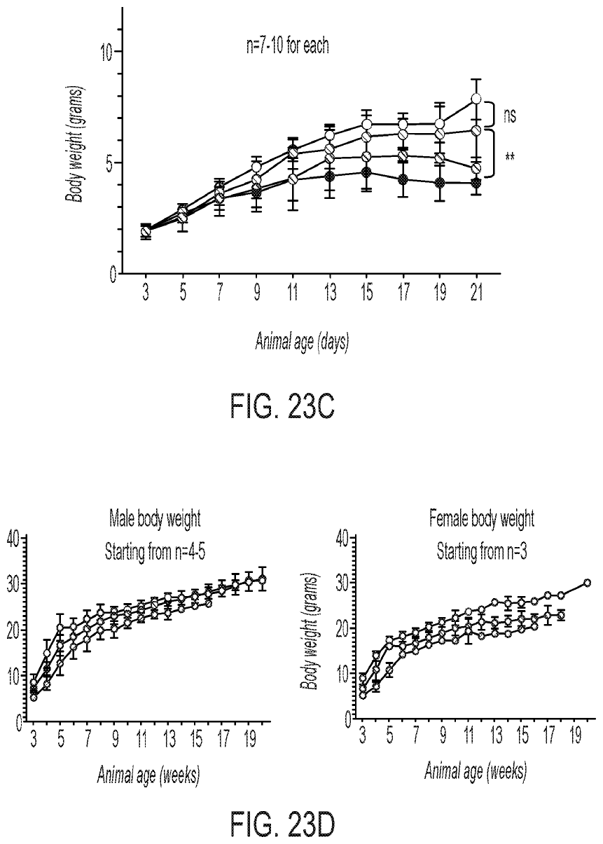

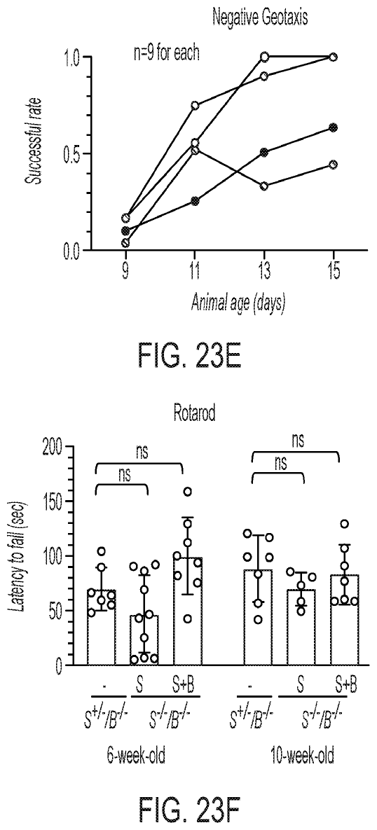

Nevertheless, survived St3gal5^{-/-}/B4galnt1^{-/-} mice receiving scAAV9-ST3GAL5.v2 treatment suffered from hindlimb clasping, a sign of motor dysfunction that was not seen in their St3gal5^{+/-}/B4galnt1^{-/-} littermate controls (FIG. 21H). One caveat of this animal model is that B4galnt1 gene knockout may exert synergistic pathological effects along with St3gal5 gene knockout, which are not readily corrected by replacing ST3GAL5 alone. To test whether B4galnt1 deficiency compromised therapeutic efficacy in St3gal5^{-/-}/B4galnt1^{-/-} mice, ssAAV9-CB-B4GALNT1 was generated and co-delivered with scAAV9-ST3GAL5.v2 by ICV administration. Indeed, this treatment regimen completely rescued lethality, growth retardation, hindlimb clasping, and motor impairment (FIGS. 23A-23G).

IV Injection of rAAV9-ST3GAL5.v2 Improved Biochemical and Phenotypic Abnormalities in GM3SD Mouse Models

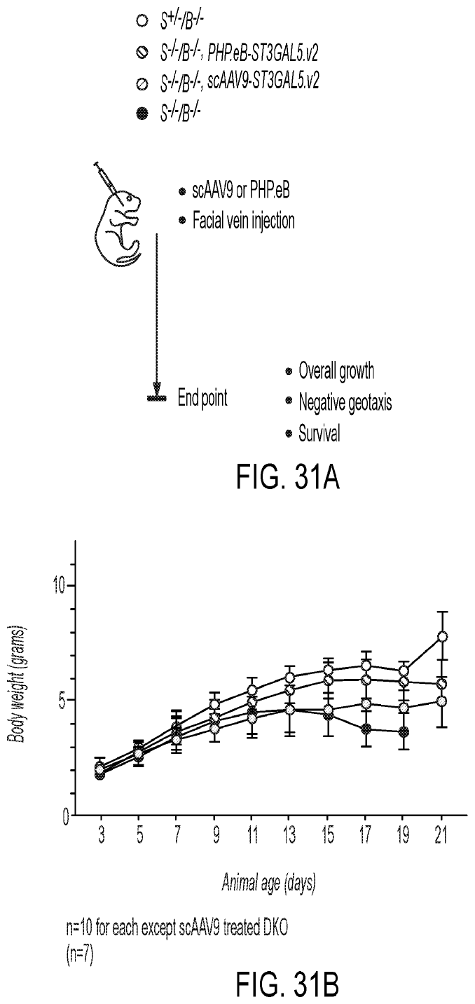

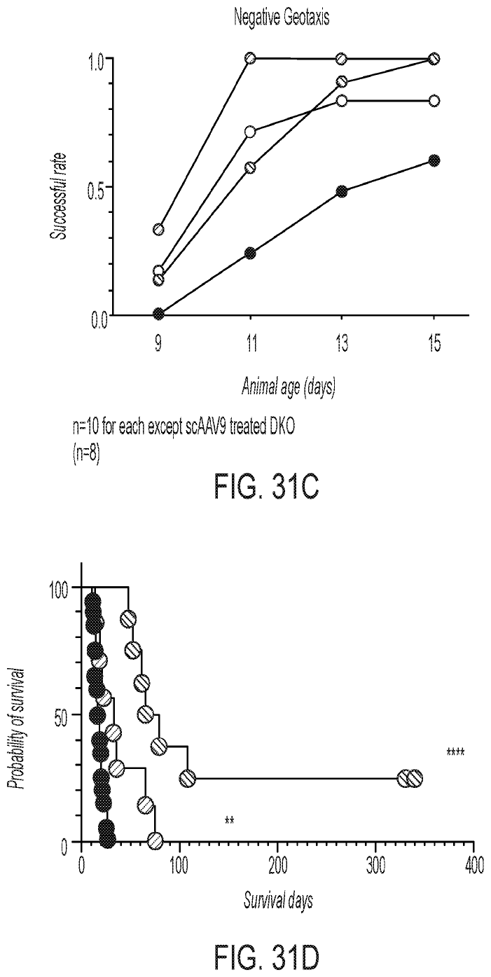

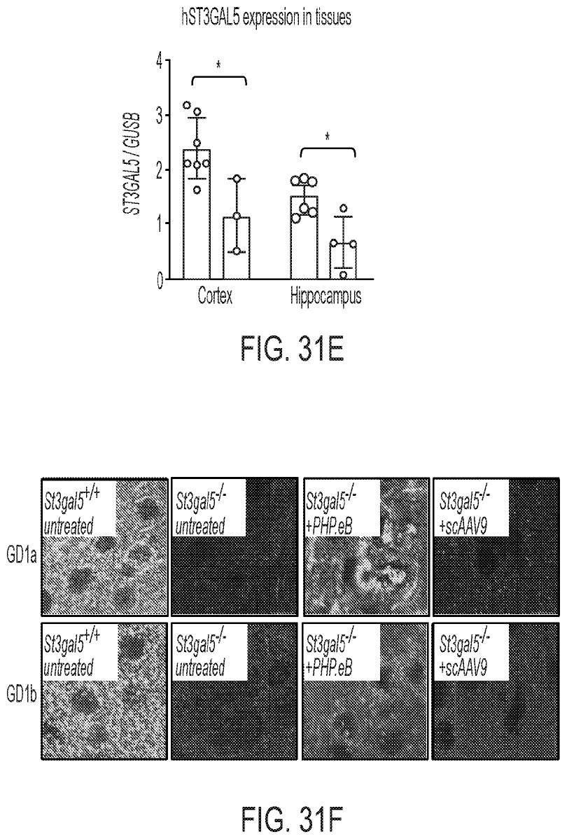

To examine whether systemically delivered scAAV9-ST3GAL5.v2 could achieve broader brain transduction and better therapeutic efficacy without causing liver toxicity, St3gal5^{-/-}/B4galnt1^{-/-} mice were treated by facial vein injection of 3×10¹¹ gc/pup at P1. Although acute lethality after administration did not occur, limited disease amelioration regarding survival (median survival: 34-days), growth, and motor function was observed (FIG. 31B-31D), likely due to low ST3GAL5 expression and poor ganglioside reconstitution in the brain as compared to ICV injection (FIG. 31E-31F). v2 construct was packaged into AAV9.PHP.eB capsid, an engineered AAV capsid that can penetrate mouse blood-brain barrier more efficiently than AAV9. Under the same systemic administration regimen, rAAV.PHP.eB-ST3GAL5.v2 led to higher transgene expression in the brain, robust ganglioside restoration, and consequently better phenotypic improvement by all measurements (FIGS. 31B-31F).

V2 constructs are packaged into AAV CAP-Mac capsid protein (see., e.g., Chuapoco et al., Intravenous gene transfer throughout the brain of infant Old World primates using AAV, bioRxiv 2022.01.08.475342). The effect of systemic administration of rAAV.CAP-MAC-ST3GAL5.v2 is tested.

### Materials and Methods

### Study Design

The primary goal of this study was to develop a recombinant adeno-associated virus (rAAV)-mediated ST3GAL5 replacement therapy to treat GM3 synthase deficiency (GM3SD). The experimental approach combined GM3SD patient derived cells and mouse models to evaluate safety, efficacy, and duration of effect. Molecular and physiological readouts include delivery of rAAV genome, ST3GAL5 expression, restoration of gangliosides, body and brain weight, motor functions, and survival. For each experiment, sample size reflected the number of independent biological replicates and was provided in the figure legends. Mice were assigned randomly to the experimental or control groups. Data from all animals were included in the analysis with no excluded outlier.

### HeLa Cell Culture and Transfection

HeLa cells were maintained in Dulbecco's Modified Eagle Medium, GlutaMAX Supplement (Gibco, Cat. No. 10569-010), supplemented with 10% (v/v) fetal bovine serum (Sigma, Cat. No. F2442) and antibiotics Penicillin-Streptomycin (100 U/ml) (Gibco, Cat. No. 15140-122) at 37° C. with 5% CO2. HeLa cells were transfected with Lipofectamine 3000 Transfection Reagent (Invitrogen, Cat. No. L3000015).

Induced Pluripotent Stem Cell (iPSC) Culture and Differentiation

iPSCs were maintained in mTESR1 (STEMCELL Technologies, Cat. No. 85850), cultured in plates pre-coated with Matrigel (Corning, Cat. No. 354277), and passaged with Rho kinase inhibitor (Abcam, Cat. No. Ab120129). Briefly, iPSCs were cultured in neural maintenance media [DMEM:F12+glutamax (Fisher Scientific, Cat. No. 10565018) and Neurobasal (Thermo Fisher Scientific, Cat. No. 21103049)], and firstly induced by neural induction media containing SB431542 (Tocris, Cat. No. 1614) and Dorsomorphin (Tocris, Cat. No. 3093) for 12 days to form the neuro-epithelial sheet. Then cells were passaged with dispase (Thermo Fisher Scientific, Cat. No. 17105041) to wells coated with laminin (Sigma-Aldrich, Cat. No. L2020) in neural maintenance medium. Cells were passaged and plated until post differentiation day 35 in the final plates pre-coated with poly-L-lysine (Sigma-Aldrich, Cat. No. P5899). Neurons were infected with lentiviral vectors in the presence of 8 μg/ml polybrene (Sigma-Aldrich, Cat. No. TR-1003-G).

### Lentiviral Vectors

Human ST3GAL5 cDNA isoforms driven by cytomegalovirus-enhancer/chicken beta-actin promoter were cloned into the lentiviral transfer plasmid pLenti-CSCGW2. The 3rd generation system was used to package lentiviral vectors. Lentivirus vector plasmid was co-transfected with packaging genome plasmids (pMDLg/Prre and pRSV/REV) and envelope plasmid (pHCMV/VSVG) to HEK293T cells using CaCl2 method. Lentivirus vector supernatants were harvested at 48 h and 72 h post-transfection and high-titer virus was concentrated via ultra-centrifugation. Virus titer was determined using QuickTiter™ Lentivirus Titer Kit (CELL BIOLABS, INC. Cat. No. VPK-107).

### Western Blot

Cell culture was lysed in ice-cold RIPA Lysis and Extraction Buffer (Thermo Fisher Scientific, Cat. No. 89901) with complete, EDTA-free protease inhibitor cocktail (Roche, Cat. No. 4693159001). Cell lysate was then sonicated. Debris was removed by centrifugation (10 minutes, 13,000 rpm, 4° C.) and supernatant was collected. Total protein concentration was determined using Bicinchoninic Acid (BCA) protein assay kit (Thermo Fisher Scientific, Cat. No. 23252). Lysates containing equal amount of total protein were boiled in Tris-Glycine SDS Sample Buffer (Invitrogen, Cat. No. LC2676) at 95° C. for 5 min. Primary antibodies rabbit anti-ST3GAL5 (Thermo Fisher Scientific, Cat. No. PA5-25730, 1:1,000 dilution), mouse anti-actin (Abcam, Cat. No. ab8226, 1:5,000 dilution) and secondary antibodies IRDye 680RD Donkey anti-Rabbit IgG (LI-COR Biosciences, Cat. No. 926-68073, 1:5,000 dilution), IRDye 800CW Donkey anti-Mouse IgG (LI-COR Biosciences, Cat. No. 926-32212, 1:5,000 dilution) were applied in Western blot. Membrane was scanned with a LI-COR Odyssey scanner (LI-COR).

### Immunofluorescence (IF) Staining

IF was performed in iPSC-derived cortical neurons and mouse brain sections. Cortical neurons were fixed with 4% paraformaldehyde (Electron Microscopy Sciences, Cat. No. 15710) after washing with Dulbecco's Phosphate-Buffered Saline (DPBS) (Thermo Fisher Scientific, Cat. No. 14190144). Following that, cells were permeabilized with 0.2% (vol/vol) Triton x-100 for neural markers or not for gangliosides staining and blocked with 5% goat serum (Invitrogen, Cat. No. 50062Z) in 0.2% (vol/vol) Triton x-100. Mouse brain was fixed in 4% paraformaldehyde at 4° C. overnight. The next day, brains were soaked in 30% sucrose at 4° C. overnight until balanced. Brains were then mounted in O.C.T. compound (Midland Scientific, Cat. No. SAKURA 4583) and stored at −80° C. until cryo-sectioning. Brain slices were permeabilized with 0.5% (vol/vol) Triton x-100 and blocked with 5% goat serum (Invitrogen, Cat. No. 50062Z). Primary antibodies, chicken anti-microtubule-associated protein 2 (MAP2) (Abcam, Cat. No. ab5392, 1:1,000 dilution), mouse anti-beta III Tubulin (Tuj1) (Abcam, Cat. No. ab78078, 1:1,000 dilution), rat anti-COUP-IF-interacting protein 2 (Ctip2) (Abcam, Cat. No. ab18465, 1:500 dilution), rabbit anti-T-box brain transcription factor 1 (Tbr1) (Abcam, Cat. No. ab31940, 1:1,000 dilution), mouse anti-ganglioside GD1a (DSHB, Cat. No. GD1a-1, 1:100 dilution), mouse anti-ganglioside GD1b (DSHB, Cat. No. GD1b01, 1:100 dilution), and mouse anti-ganglioside GT1b (DSHB, Cat. No. GT1b-1, 1:100 dilution) were used in immunodetection in blocking buffer at 4° C. overnight. Secondary antibodies goat anti-chicken IgY H&L, Alexa Fluor 488 (Abcam, Cat. No. ab150169, 1:1,000 dilution), donkey anti-mouse IgG H&L, Alexa Fluor 594 (Abcam, Cat. No. ab150108, 1:1,000 dilution), goat anti-rat IgG H&L, Alexa Fluor 647 (Abcam, Cat. No. ab150167, 1:1,000 dilution), goat anti-rabbit IgG H&L, Alexa Fluor 488 (Abcam, Cat. No. ab150077, 1:1,000 dilution) and goat anti-mouse IgG H&L, Alexa Fluor 488 (Thermo Fisher, Cat. No. A11029) were incubated within blocking buffer at room temperature for one hour. Sliced were mounted using Prolong Diamond Antifade Mountant with DAPI (Fisher scientific, Cat. No. P36962). Images were taken on a Leica TCS SP8 confocal microscope. Quantification of GD1a and GD1b was performed using the ImageJ software.

### Adeno-Associated Virus (AAV) Vectors

Human ST3GAL5 cDNA isoforms driven by cytomegalovirus-enhancer/chicken beta-actin promoter and human ST3GAL5 cDNA isoforms plus miR122 binding sites driven by Synapsin1 promoter were cloned into AAV plasmids. The plasmids were sequenced throughout the expression cassette, and the integrity of inverted terminal repeats (ITR) was confirmed by restriction enzyme digestion. AAV vectors were produced by transient triple transfection in HEK293 cells and purified by CsCl gradient sedimentation for AAV9 or by iodixanol gradient sedimentation for PHP.eB vectors. Vector titers were determined by droplet digital PCR and vector purity was assessed by gel electrophoresis followed by silver staining.

### Animal Use

St3gal54−/B4galnt1^{+/-} males were imported from Regeneron Pharmaceuticals, Inc. and bred with C57BL6NTac female (TACONIC, B6-F). Newborns were genotyped at the date of birth. Briefly, 1 mm tail tips were cut. Genomic DNA was extracted by boiling in 25 mM NaOH+0.4 mM EDTA (pH 8.0) at 100° C. for 90 minutes, followed by mixing with 40 mM Tris-Hcl (pH 8.0). St3gal5 and B4galnt1 genes were determined by quantitative PCR (qPCR) using Taqman reagents targeting St3gal5 (Thermo Fisher Scientific, Assay ID: APH6DZ6, 9057mTGU; Assay ID: APMFZ6Z, 9057mTGD), B4galnt1 (LGC Biosearch Technologies, Cat. No. DLOM-RFB-5, Assay ID: 15582TU; Assay ID: LacZ) and Tfrc (Thermo Fisher Scientific, Cat. No. 4458367). To harvest tissues, mice were anesthetized with isoflurane and transcardially perfused with ice-cold PBS. Tissues were immediately dissected, snap-frozen in liquid nitrogen, and stored at −80° C. Facial vein injections were performed on postnatal day 1 (P1) via right facial vein at 3×10¹¹ genome copies (GC) per pup. Intracerebroventricular (I.C.V.) injections were performed on P1 at 3×10¹⁰ GC bilaterally per pup. After procedure, pups were cleaned with 70% ethanol and rubbed with bedding material.

DNA/RNA Extraction, Quantitative Realtime PCR (qPCR) and Droplet Digital PCR (ddPCR)

Total DNA and RNA were extracted from snap frozen mouse tissues using AllPrep DNA/RNA Mini kit (Qiagen, Cat. No. 80204). Viral vector genome copy number was determined in a multiplexed reaction using ddPCR Supermix for Probes (No dUTP) (Bio-Rad, Cat. No. 1863024) and Taqman reagents targeting ST3GAL5 (Thermo Fisher Scientific, Assay ID: APGZHGD) and Tfrc (Thermo Fisher Scientific, Cat. No. 4458367). One g of total RNA was reverse transcribed into cDNA using High-capacity cDNA Reverse Transcription Kit (Applied Biosystems, Cat. No. 4368813). Exogenous human ST3GAL5 and mouse St3gal5 cDNA were quantified in a multiplexed reaction using Taqman reagents targeting ST3GAL5 (Thermo Fisher Scientific, Assay ID: APGZHGD), St3gal5 (Thermo Fisher Scientific, Assay ID: Mm00488232_m1) and Gusb (Thermo Fisher Scientific, Assay ID: Mm01197698_m1). ddPCR was performed with a QX200 ddPCR system (Bio-Rad). qPCR was performed on a ViiA 7 Real-Time PCR system using Taqman Gene expression master mix (Thermo Fisher Scientific, Cat. No. 4369016) and Taqman reagents targeting Chop (Thermo Fisher Scientific, Assay ID: Mm01135937_g1) and Tnfa (Thermo Fisher Scientific, Assay ID: Mm00443260_g1).

### Mass Spectrometry (MS)

Brain tissue samples were homogenized in water (4 mL/g wet tissue) using an Omni Bead Ruptor (Cole-Parmer, Cat No. Mfr19-628). The LacCer, GM1, GM2, GM3 were extracted from 50 uL of homogenate or serum with 200 uL of methanol containing d3-Lc (16:0) (Matreya LLC, Cat. No, 1534), d3-GM1 (18:0) (Matreya LLC, Cat. No. 2050), d3-GM2 (18:0) (Matreya LLC, Cat. No, 2051), and d3-GM3 (18:0) (Matreya LLC, Cat. No. 2052) as the internal standards for LacCer, GM1, GM2, GM3, respectively. Quality control (QC) samples were prepared by pooling aliquots of positive samples and injected every five study samples to monitor instrument performance throughout these analyses. The analysis of LacCer, GM1, GM2, GM3 was performed on a Shimadzu 20AD HPLC system and a SIL-20AC autosampler coupled to 6500QTRAP+mass spectrometer (AB Sciex) operated in positive multiple reaction monitoring mode. Data processing was conducted with Analyst 1.6.3 (Applied Biosystems). The relative quantification data for all analytes were presented as the peak ratios of analytes to their internal standard.

### Mouse Monitoring and Behavioral Assays

Mice were blindly weighed every other day until weaning at 21 days old. After weaning, each mouse was weighed and evaluated weekly by a trained observer for adverse events.

### Negative Geotaxis

Negative geotaxis assay was examined every other day for P9-P15 pups on a 45° incline plane. Prior to the test, animals were placed on the plane to acclimate for one minute. Mouse head was facing downwards, success was marked when mouse rotated 180° to the head-up position while failure was when mouse dropped off from the plane. The ability of finishing the assay was recorded. Each mouse was tested for three times and the success rate of completing the assay was plotted.

### Accelerated Rotarod

Coordinated motor functions were examined in treated mice and littermates using the 4-40 rpm accelerating rotarod test. Mice were tested at six weeks old and ten weeks old. Tested mice were trained two days before the testing day for three tests each. Prior to the test, the animals were placed on the rotarod machine to acclimate for at least one minute. Each mouse was tested for three times and the best latency to fall was recorded and plotted.

### Histology and Immunohistochemistry (IHC)

Mouse brain and liver were fixed in 10% formalin (Fisher Scientific, Cat. No. SF100-20). Paraffin embedding, sectioning, hematoxylin and eosin (H&E) staining, terminal deoxynucleotidyl transferase dUTP nick end labelling (TUNEL) staining (Roche, Cat. No. 11684817910) and IHC were performed by the Morphology Core at University of Massachusetts Chan Medical School under standard conditions. Mouse anti-GFAP antibody (EMD Millipore, Cat. No. MAB360, 1:500 dilution) was used in IHC. Images were taken on a Leica DM5500 B microscope. The quantification of GFAP IHC was performed using the Image FIJI software as previously described.

### ProcartaPlex Multiplex Immunoassays

Total protein was extracted in ice-cold RIPA Lysis and Extraction Buffer (Thermo Fisher Scientific, Cat. No. 89901) with complete, EDTA-free protease inhibitor cocktail (Roche, Cat. No. 4693159001) from snap frozen tissues. Protein concentration was determined using Bicinchoninic Acid (BCA) protein assay kit (Thermo Fisher Scientific, Cat. No. 23252). Normalized protein extracts were loaded on procartaplex mix&match panel (Thermo Fisher Scientific). Values were acquired by Bio-Plex MAGPIX (Bio-Rad).

### Messenger RNA Sequencing (RNA-Seq)

RNA-seq was carried out by Novegene (Novogene Corporation Inc, CA) under standard conditions. RNA was extracted using Trizol phase separation method from cell debris. Isolated RNA sample integrity and concentration was assessed by Agilent bioanalyzer 2100. A total amount of 1 μg RNA per sample was used as input material for RNA sample preparations. Sequencing libraries were generated using NEBNext® Ultra RNA Library Prep Kit for Illumina® (New England BioLabs, Cat. No. E7770L) following manufacturer's recommendations. Briefly, mRNA was purified from total RNA using poly-T oligo-attached magnetic beads. Fragmentation was carried out using divalent cations under elevated temperature in NEBNext First Strand Synthesis Reaction Buffer (5×) (New England BioLabs). First strand cDNA was synthesized using random hexamer primer and M-MuL V Reverse Transcriptase (RNase H-). Second strand cDNA synthesis was subsequently performed using DNA Polymerase I and RNase H. Final libraries quantity was assessed on the Agilent Bioanalyzer 2100 system. The clustering of the index-coded samples were performed on a cBot Cluster Generation System using PEE Cluster Kit cBot-HS (Illumina, California, USA) according to the manufacturer's instructions. After cluster generation, the library preparations were sequenced on an Illumina NovaSeq 6000 platform and paired-end reads were generated.

For data analysis, 3′ adapter sequence was removed using Trimmomatic (with parameters ILLUMINACLIP, min_length, 10; seed mismatches, 2; palindrome clip threshold, 30; simple clip threshold, 5). Then, reads were mapped to mouse_mm10_gencode_using STAR. To estimate expression levels, RSEM55 was used to align reads to a predefined set of transcripts from GENCODE. Finally, the RSEM quantification matrix, i.e., estimated counts for each gene and/or for each annotated isoform, was used for differential gene expression analysis. Count matrix was loaded into DEBrowser software for interactive analysis. Data analysis was performed on the RNA-seq pipeline of the DolphinNext.

### Statistical Analysis

All data are presented as mean SD and analyzed using GraphPad Prism software (Version 9). Two-sided student t-test was used to compare two groups, and one-way analysis of variance (ANOVA) was used to compare among multiple groups. Animal weight was analyzed by two-way ANOVA and survival was analyzed by Log-rank (Mantel-Cox) test. *p<0.05, **p<0.01, ***p<0.001, ****p<0.0001, ns: not significant.

### SEQUENCE LISTING

The skilled artisan recognizes that certain sequences in the Sequence Listing are represented as linear nucleic acid sequences corresponding to circular plasmid sequences. Accordingly, in some embodiments, sequences described herein represent a contiguous polynucleotide (e.g., sequences sharing a continuous phosphate backbone), such that the first base and the last base of the linear representation are positioned next to one another. The Sequence listing contains the sequences as shown below:
>construct #1_pAAVCBA opt_FL_h ST3GAL5-1a-2 (SEQ ID NO: 1)
>construct #3_pAAVCBA Opt_M2_hST3GAL51a-2 (SEQ ID NO: 3)
>construct #4_pAAVCBA Opt_M3_hST3GAL5-1a-2 (SEQ ID NO: 4)
>construct #5_pAAVCBA_Opt_KM3_hST3GAL5-1a-2 (SEQ ID NO: 5)
>construct #6_pAAVCBA FL_hST3GAL5-1a-2 (SEQ ID NO: 6)
>construct #7_pAAVCBA FL_opt_M3_hST3GAL5-1a-2 (SEQ ID NO: 7)
>Human GM3S Ia Type 2 isoform M1 variant amino acid sequence (SEQ ID NO: 8)
>Human GM3S Ia Type 2 isoform M2 variant amino acid sequence (SEQ ID NO: 9)
>Human GM3S Ia Type 2 isoform M3 variant amino acid sequence (SEQ ID NO: 10)
>construct #2_miR122 BS pAAV CBA opt_M1_hST3GAL5-1a-2_mir122 binding (SEQ ID NO: 11)
>construct #3_miR122 BS pAAV CBA opt_M2_hST3GAL5-1a-2_mir122 binding (SEQ ID NO: 12)
>construct #4_miR122 BS pAAV CBA opt_M3_hST3GAL5-1a-2_mir122 binding (SEQ ID NO: 13)
>construct #5_miR122 BS pAAV CBA opt_KM3_hST3GAL5-1a-2_mir122 binding (SEQ ID NO: 14)
>pAAV_hSyn1_EGFP_miR122BS (SEQ ID NO: 15)
>pAAV_hSyn1_intron_EGFP_miR122BS (SEQ ID NO: 16)
pAAV_hSyn1_intron_opt_KM3_hST3GAL5-1a-2_miR122BS (SEQ ID NO: 17)
>pAAV_hSyn1_intron_opt_M1_hST3GAL5-1a-2_miR122BS (SEQ ID NO: 18)
>pAAV_hSyn1_intron_opt_M2_hST3GAL5-1a-2_miR122BS (SEQ ID NO: 19)
>pAAV_hSyn1_intron_opt_M3_hST3GAL5-1a-2_miR122BS (SEQ ID NO: 20)
>pAAV_hSyn1_opt_KM3_hST3GAL5-1a-2 (SEQ ID NO: 21)
>pAAV_hSyn1_opt_KM3_hST3GAL5-1a-2_miR122BS (SEQ ID NO: 22)
>pAAV_hSyn1_opt_M1_hST3GAL5-1a-2 (SEQ ID NO: 23)
>pAAV_hSyn1_opt_M1_hST3GAL5-1a-2_miR122BS (SEQ ID NO: 24)
>pAAV_hSyn1_opt_M2_hST3GAL5-1a-2 (SEQ ID NO: 25)
>pAAV_hSyn1_opt_M2_hST3GAL5-1a-2_miR122BS (SEQ ID NO: 26)
>pAAV_hSyn1_opt_M3_hST3GAL5-1a-2 (SEQ ID NO: 27)
>pAAV_hSyn1_opt_M3_hST3GAL5-1a-2_miR122BS (SEQ ID NO: 28)
>pAAV_phST3GAL5_177_EGFP (SEQ ID NO: 29)
>pAAV_phST3GAL5_177_KM3_hST3GAL5-1a-2 (SEQ ID NO: 30)
>pAAV_phST3GAL5_177_M1_hST3GAL5-1a-2 (SEQ ID NO: 31)
>pAAV_phST3GAL5_177_M2_hST3GAL5-1a-2 (SEQ ID NO: 32)
>pAAV_phST3GAL5_177_M3_hST3GAL5-1a-2 (SEQ ID NO: 33)
>pAAV_phST3GAL5_1600_EGFP (SEQ ID NO: 34)
>pAAV_phST3GAL5_1600_KM3_hST3GAL5-1a-2 (SEQ ID NO: 35)
>pAAV_phST3GAL5_1600_M1_hST3GAL5-1a-2 (SEQ ID NO: 36)
>pAAV_phST3GAL5_1600_M2_hST3GAL5-1a-2 (SEQ ID NO: 37)
>pAAV_phST3GAL5_1600_M3_hST3GAL5-1a-2 (SEQ ID NO: 38)
>Wild-type Human GM3S Ia Type 2 isoform M1 variant coding sequence_codon un-optimized (SEQ ID NO: 39)
>Human GM3S Ia Type 2 isoform M1 variant coding sequence_codon optimized (SEQ ID NO: 40)
>Human GM3S Ia Type 2 isoform M2 variant coding sequence_codon optimized (SEQ ID NO: 41)
>Human GM3S Ia Type 2 isoform M3 variant coding sequence_codon optimized (SEQ ID NO: 42)
>Human GM3S Ia Type 2 isoform KM3 variant coding sequence_codon optimized (SEQ ID NO: 43)

1. A method for treating a GM3 synthase (GM3S) deficiency in a subject in need thereof, the method comprising administering to a subject having a GM3 synthase (GM3S) deficiency a recombinant adeno-associated virus (rAAV) via intracerebroventricular (ICV) administration, wherein the rAAV comprises:
(i) a capsid protein; and
(ii) an isolated nucleic acid comprising a transgene having a nucleic acid sequence encoding one or more monosialodihexosylganglioside synthase (GM3S) protein isoforms, wherein the transgene is flanked by adeno-associated virus (AAV) inverted terminal repeats (ITRs).

2. (canceled)

3. The method of claim 1, wherein the transgene encodes a one or more GM3S Ia Type 1 isoforms.

4. (canceled)

5. The method of claim 3, wherein the one or more GM3S Ia Type 1 isoforms are translated from an M2 initiation codon or an M3 initiation codon.

6. (canceled)

7. The method of claim 1, wherein the one or more GM3S isoforms are GM3S Ia Type 2 isoforms.

8. (canceled)

9. The method of claim 7, wherein the one or more GM3S Ia Type 2 isoforms are translated from an M3 initiation codon, an M2 initiation codon or an M1 initiation codon.

10. (canceled)

11. The method of claim 1, wherein the transgene encodes one or more GM3S Ib isoforms.

12. (canceled)

13. The method of claim 11, wherein the one or more GM3S Ib isoforms are GM3S Ib Type 1 isoforms and are translated from an M2 initiation codon or an M3 initiation codon.

14. (canceled)

15. The method of claim 11, wherein the one or more GM3S Ib isoforms are GM3S Ib Type 2 isoforms, and are translated from an M2 initiation codon or an M3 initiation codon.

16-17. (canceled)

18. The method of claim 1, wherein the transgene encodes one or more GM3S Ic isoforms.

19. The method of claim 18, wherein the one or more GM3S Ic isoforms are translated from an M2′ initiation codon, an M2 initiation codon, or an M3 initiation codon.

20-53. (canceled)

54. A recombinant adeno associated virus (rAAV) comprising
(i) an AAV Cap-Mac capsid protein; and
(ii) an isolated nucleic acid comprising a transgene having a nucleic acid sequence encoding one or more monosialodihexosylganglioside synthase (GM3S) Ia Type 2 protein isoforms, and wherein the transgene is flanked by adeno-associated virus (AAV) inverted terminal repeats (ITRs).

55. The rAAV of claim 54, wherein the one or more GM3S Ia Type 2 isoforms are translated from an M1 initiation codon, an M2 initiation codon, or an M3 initiation codon.

56-57. (canceled)

58. The rAAV of claim 54, wherein the transgene further comprises a Kozak sequence (GCCACC) operably linked to the nucleic acid sequence encoding the one or more GM3S protein isoforms.

59. The rAAV of claim 54, wherein the each of the one or more GM3S protein isoforms comprises or consists of the amino acid sequence set forth in any one of SEQ ID NOs. 8-10.

60. The rAAV of claim 54, wherein the transgene further comprises a promoter operably linked to the nucleic acid sequence encoding the one or more GM3 synthase protein isoforms, optionally wherein the promoter is a chicken beta-actin (CBA) promoter, a hST3GAL5 promoter, or a Synapsin I (Syn1) promoter.

61-67. (canceled)

68. The rAAV of claim 54, wherein the isolated nucleic acid comprises the sequence set forth in any one of SEQ ID NOs: 1-7, and 11-43.

69. The rAAV of claim 54, further comprising one or more miRNA binding sites.

70-72. (canceled)

73. The rAAV of claim 54, wherein the rAAV is a self-complementary rAAV.

74. The rAAV of claim 55, wherein the rAAV encodes one or more GM3S Ia Type 2 isoforms are translated from an M3 initiation codon.

75. The rAAV of claim 74, wherein the transgene further comprises a Kozak sequence (GCCACC) operably linked to the nucleic acid sequence encoding the one or more GM3S Ia Type 2 isoforms translated from an M3 initiation codon.


SEQUENCE LISTING (48 sequences)

SEQ ID NO: 1 - DNA, 3732 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccacgcgcccctcctccgcatctgagcgggggagcggcggcccccagctgaatgggcgcgagagcggcgctgggggcgggtgggggcgcggggtaccgggctggcggccggccggcgccccctcattagtatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 3603 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - DNA, 3519 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - DNA, 3419 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgacctggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 5 - DNA, 3441 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 3732 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccacgcgcccctcctccgcatctgagcgggggagcggcggcccccagctgaatgggcgcgagagcggcgctgggggcgggtgggggcgcggggtaccgggctggcggccggccggcgccccctcattagtatgcggacgaaggcggcgggctgcgcggagcggcgtcccctgcagccgcggaccgaggcagcggcggcacctgccggccgagcaatgccaagtgagtacacctatgtgaaactgagaagtgattgctcgaggccttccctgcaatggtacacccgagctcaaagcaagatgagaaggcccagcttgttattaaaagacatcctcaaatgtacattgcttgtgtttggagtgtggatcctttatatcctcaagttaaattatactactgaagaatgtgacatgaaaaaaatgcattatgtggaccctgaccatgtaaagagagctcagaaatatgctcagcaagtcttgcagaaggaatgtcgtcccaagtttgccaagacatcaatggcgctgttatttgagcacaggtatagcgtggacttactcccttttgtgcagaaggcccccaaagacagtgaagctgagtccaagtacgatcctccttttgggttccggaagttctccagtaaagtccagaccctcttggaactcttgccagagcacgacctccctgaacacttgaaagccaagacctgtcggcgctgtgtggttattggaagcggaggaatactgcacggattagaactgggccacaccctgaaccagttcgatgttgtgataaggttaaacagtgcaccagttgagggatattcagaacatgttggaaataaaactactataaggatgacttatccagagggcgcaccactgtctgaccttgaatattattccaatgacttatttgttgctgttttatttaagagtgttgatttcaactggcttcaagcaatggtaaaaaaggaaaccctgccattctgggtacgactcttcttttggaagcaggtggcagaaaaaatcccactgcagccaaaacatttcaggattttgaatccagttatcatcaaagagactgcctttgacatccttcagtactcagagcctcagtcaaggttctggggccgagataagaacgtccccacaatcggtgtcattgccgttgtcttagccacacatctgtgcgatgaagtcagtttggcgggttttggatatgacctcaatcaacccagaacacctttgcactacttcgacagtcaatgcatggctgctatgaactttcagaccatgcataatgtgacaacggaaaccaagttcctcttaaagctggtcaaagagggagtggtgaaagatctcagtggaggcattgatcgtgaattttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 3732 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccacgcgcccctcctccgcatctgagcgggggagcggcggcccccagctgaatgggcgcgagagcggcgctgggggcgggtgggggcgcggggtaccgggctggcggccggccggcgccccctcattagtatgcggacgaaggcggcgggctgcgcggagcggcgtcccctgcagccgcggaccgaggcagcggcggcacctgccggccgagcaatgccaagtgagtacacctatgtgaaactgagaagtgattgctcgaggccttccctgcaatggtacacccgagctcaaagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - protein, 418 aa, Artificial Sequence
  Synthetic Polypeptide
MRTKAAGCAERRPLQPRTEAAAAPAGRAMPSEYTYVKLRSDCSRPSLQWYTRAQSKMRRPSLLLKDILKCTLLVFGVWILYILKLNYTTEECDMKKMHYVDPDHVKRAQKYAQQVLQKECRPKFAKTSMALLFEHRYSVDLLPFVQKAPKDSEAESKYDPPFGFRKFSSKVQTLLELLPEHDLPEHLKAKTCRRCVVIGSGGILHGLELGHTLNQFDVVIRLNSAPVEGYSEHVGNKTTIRMTYPEGAPLSDLEYYSNDLFVAVLFKSVDFNWLQAMVKKETLPFWVRLFFWKQVAEKIPLQPKHFRILNPVIIKETAFDILQYSEPQSRFWGRDKNVPTIGVIAVVLATHLCDEVSLAGFGYDLNQPRTPLHYFDSQCMAAMNFQTMHNVTTETKFLLKLVKEGVVKDLSGGIDREF

SEQ ID NO: 9 - protein, 390 aa, Artificial Sequence
  Synthetic Polypeptide
MPSEYTYVKLRSDCSRPSLQWYTRAQSKMRRPSLLLKDILKCTLLVFGVWILYILKLNYTTEECDMKKMHYVDPDHVKRAQKYAQQVLQKECRPKFAKTSMALLFEHRYSVDLLPFVQKAPKDSEAESKYDPPFGFRKFSSKVQTLLELLPEHDLPEHLKAKTCRRCVVIGSGGILHGLELGHTLNQFDVVIRLNSAPVEGYSEHVGNKTTIRMTYPEGAPLSDLEYYSNDLFVAVLFKSVDFNWLQAMVKKETLPFWVRLFFWKQVAEKIPLQPKHFRILNPVIIKETAFDILQYSEPQSRFWGRDKNVPTIGVIAVVLATHLCDEVSLAGFGYDLNQPRTPLHYFDSQCMAAMNFQTMHNVTTETKFLLKLVKEGVVKDLSGGIDREF

SEQ ID NO: 10 - protein, 362 aa, Artificial Sequence
  Synthetic Polypeptide
MRRPSLLLKDILKCTLLVFGVWILYILKLNYTTEECDMKKMHYVDPDHVKRAQKYAQQVLQKECRPKFAKTSMALLFEHRYSVDLLPFVQKAPKDSEAESKYDPPFGFRKFSSKVQTLLELLPEHDLPEHLKAKTCRRCVVIGSGGILHGLELGHTLNQFDVVIRLNSAPVEGYSEHVGNKTTIRMTYPEGAPLSDLEYYSNDLFVAVLFKSVDFNWLQAMVKKETLPFWVRLFFWKQVAEKIPLQPKHFRILNPVIIKETAFDILQYSEPQSRFWGRDKNVPTIGVIAVVLATHLCDEVSLAGFGYDLNQPRTPLHYFDSQCMAAMNFQTMHNVTTETKFLLKLVKEGVVKDLSGGIDREF

SEQ ID NO: 11 - DNA, 6496 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 12 - DNA, 6412 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 13 - DNA, 6328 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 14 - DNA, 6334 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 15 - DNA, 4725 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcctactagacgcg

SEQ ID NO: 16 - DNA, 4967 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcctactagacgcg

SEQ ID NO: 17 - DNA, 5318 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 18 - DNA, 5480 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 19 - DNA, 5396 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 20 - DNA, 5311 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 21 - DNA, 5004 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 22 - DNA, 5076 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 23 - DNA, 5166 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 24 - DNA, 5238 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 25 - DNA, 5082 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 26 - DNA, 5154 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 27 - DNA, 4998 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 28 - DNA, 5070 bp, Artificial Sequence
  Synthetic Polynucleotide
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 29 - DNA, 4372 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcctactagacgcg

SEQ ID NO: 30 - DNA, 4729 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 31 - DNA, 4891 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 32 - DNA, 4807 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 33 - DNA, 4723 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 34 - DNA, 5796 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcctactagacgcg

SEQ ID NO: 35 - DNA, 6153 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 36 - DNA, 6315 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 37 - DNA, 6231 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 38 - DNA, 6147 bp, Artificial Sequence
  Synthetic Polynucleotide
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 39 - DNA, 1257 bp, Artificial Sequence
  Synthetic Polynucleotide
atgcggacgaaggcggcgggctgcgcggagcggcgtcccctgcagccgcggaccgaggcagcggcggcacctgccggccgagcaatgccaagtgagtacacctatgtgaaactgagaagtgattgctcgaggccttccctgcaatggtacacccgagctcaaagcaagatgagaaggcccagcttgttattaaaagacatcctcaaatgtacattgcttgtgtttggagtgtggatcctttatatcctcaagttaaattatactactgaagaatgtgacatgaaaaaaatgcattatgtggaccctgaccatgtaaagagagctcagaaatatgctcagcaagtcttgcagaaggaatgtcgtcccaagtttgccaagacatcaatggcgctgttatttgagcacaggtatagcgtggacttactcccttttgtgcagaaggcccccaaagacagtgaagctgagtccaagtacgatcctccttttgggttccggaagttctccagtaaagtccagaccctcttggaactcttgccagagcacgacctccctgaacacttgaaagccaagacctgtcggcgctgtgtggttattggaagcggaggaatactgcacggattagaactgggccacaccctgaaccagttcgatgttgtgataaggttaaacagtgcaccagttgagggatattcagaacatgttggaaataaaactactataaggatgacttatccagagggcgcaccactgtctgaccttgaatattattccaatgacttatttgttgctgttttatttaagagtgttgatttcaactggcttcaagcaatggtaaaaaaggaaaccctgccattctgggtacgactcttcttttggaagcaggtggcagaaaaaatcccactgcagccaaaacatttcaggattttgaatccagttatcatcaaagagactgcctttgacatccttcagtactcagagcctcagtcaaggttctggggccgagataagaacgtccccacaatcggtgtcattgccgttgtcttagccacacatctgtgcgatgaagtcagtttggcgggttttggatatgacctcaatcaacccagaacacctttgcactacttcgacagtcaatgcatggctgctatgaactttcagaccatgcataatgtgacaacggaaaccaagttcctcttaaagctggtcaaagagggagtggtgaaagatctcagtggaggcattgatcgtgaattttga

SEQ ID NO: 40 - DNA, 1257 bp, Artificial Sequence
  Synthetic Polynucleotide
atgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttga

SEQ ID NO: 41 - DNA, 1173 bp, Artificial Sequence
  Synthetic Polynucleotide
atgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttga

SEQ ID NO: 42 - DNA, 1089 bp, Artificial Sequence
  Synthetic Polynucleotide
atgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttga

SEQ ID NO: 43 - DNA, 1095 bp, Artificial Sequence
  Synthetic Polynucleotide
gccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttga

SEQ ID NO: 44 - RNA, 10 nt, Artificial Sequence
  Synthetic
auuaguaugc

SEQ ID NO: 45 - DNA, 28 bp, Artificial Sequence
  Synthetic
ccattctgggtacgactcttcttttgga

SEQ ID NO: 46 - DNA, 27 bp, Artificial Sequence
  Synthetic
ccattctgggtacgactcttcttttgg

SEQ ID NO: 47 - DNA, 30 bp, Artificial Sequence
  Synthetic
ccattctgggtatgactcttcttttggaag

SEQ ID NO: 48 - DNA, 27 bp, Artificial Sequence
  Synthetic
ccattctgggtatgactcttcttttgg